(19) United States
(12) Patent Application Publication
(10) Pub. No.: US 2025/0059520 A1
(43) Pub. Date: Feb. 20, 2025

(54) SGK1 INHIBITORY COMPOSITIONS AND METHODS

(71) Applicant: Arizona Board of Regents on Behalf of the University of Arizona, Tucson, AZ (US)

(72) Inventors: Erik Blackwood, Phoenix, AZ (US); Christopher Glembotski, Phoenix, AZ (US)

(21) Appl. No.: 18/718,521

(22) PCT Filed: Dec. 12, 2022
(86) PCT No.: PCT/US2022/081357

§ 371 Date: Jun. 11, 2024

Related U.S. Application Data
(60) Provisional application No. 63/289,399, filed on Dec. 14, 2021.

(51) Int. Cl. C12N 9/12; A61K 38/00; C07K 16/40

(52) U.S. Cl. CPC C12N 9/12; C07K 16/40; A61K 38/00; C07K 2319/10; C12Y 207/11001

(57) ABSTRACT

Disclosed herein is a fusion peptide comprising an inactive peptide fragment of Serum/Glucocorticoid Regulated Kinase 1 (SGK1) and a cell internalization sequence, wherein the SGK1 fragment is capable of binding and sequestering glucocorticoid-induced leucine zipper (GILZ). In some embodiments, the inactive peptide fragment of SGK1 binds and sequesters GILZ, preventing it from binding and extending the half-life of endogenous SGK1. Also disclosed herein is a method for treating a disease associated with aberrant Serum/Glucocorticoid Regulated Kinase 1 (SGK1) activity in a subject that involves administering to the subject an agent that blocks the binding of endogenous SGK1 to endogenous glucocorticoid-induced leucine zipper (GILZ).

### CROSS-REFERENCE TO RELATED APPLICATIONS

This application claims benefit of U.S. Provisional Application No. 63/289,399, filed Dec. 14, 2021, which is hereby incorporated herein by reference in its entirety.

### STATEMENT REGARDING FEDERALLY SPONSORED RESEARCH OR DEVELOPMENT

This invention was made with Government Support under Grant No. HL157027 awarded by the National Institutes of Health. The Government has certain rights in the invention.

### SEQUENCE LISTING

This application contains a sequence listing filed in ST.26 format entitled “220111_2200_Sequence_Listing” created on Nov. 22, 2022. The content of the sequence listing is incorporated herein in its entirety.

### BACKGROUND

Serum and glucocorticoid-inducible kinase 1 (SGK1) is an AGC kinase that has been reported to be involved in a variety of physiological and pathological processes. Recent evidence has accumulated that SGK1 acts as an essential Akt-independent mediator of phosphatidylinositol 3-kinase (PI3K)/mammalian target of rapamycin (mTOR) signaling pathway in cancer. SGK1 is overexpressed in several tumors, including prostate cancer, colorectal carcinoma, glioblastoma, breast cancer, and endometrial cancer. The functions of SGK1 include regulating tumor growth, survival, metastasis, autophagy, immunoregulation, calcium (Ca2+) signaling, cancer stem cells, cell cycle, and therapeutic resistance. Safe and effective inhibitors of SGK1 are therefore needed to treat a wide variety of diseases and disorders.

### SUMMARY

Disclosed herein is a fusion peptide comprising an inactive peptide fragment of Serum/Glucocorticoid Regulated Kinase 1 (SGK1) and a cell internalization sequence, wherein the SGK1 fragment is capable of binding and sequestering glucocorticoid-induced leucine zipper (GILZ). In some embodiments, the inactive peptide fragment of SGK1 binds and sequesters GILZ, preventing it from binding and extending the half-life of endogenous SGK1.

Therefore, disclosed herein is a fusion peptide containing a peptide fragment of SGK1 comprising at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 consecutive amino acids of VFYAVKVLQKKAILKKKEEKHIMSERNVLLKNVKHP (SEQ ID NO:1; aa 122-157 of SGK1) and an internalization sequence.

In some embodiments, the peptide fragment of SGK1 lacks at least amino acids 1-97 of SGK1 so that it binds GILZ but does not lack kinase activity.

In some embodiments, the amino acid sequence for human SGK1 has the amino acid sequence SEQ ID NO:2. Therefore, in some embodiments, the peptide fragment of SGK1 contains less than 37, 38, 39, 40, 41, 42, 43, 44, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, or 400 consecutive amino acids from SEQ ID NO:2.

In some embodiments, the fusion peptide contains 2 or more SGK1 fragments, including 2, 3, 4, 5, 6, 7, 8, 9, 10 or more SGK1 fragments. These fragments can in some embodiments, be separated by a linker.

In some embodiments, the internalization sequence comprises a HIV-TAT internalization domain, such as the amino acid sequence SEQ ID NO:5. Therefore, in some embodiments, the fusion peptide comprises or consists essentially the amino acid sequence SEQ ID NO:20.

Also disclosed herein is a method for treating a disease associated with aberrant Serum/Glucocorticoid Regulated Kinase 1 (SGK1) activity in a subject that involves administering to the subject an agent that blocks the binding of endogenous SGK1 to endogenous glucocorticoid-induced leucine zipper (GILZ). For example, in some embodiments, the agent is a fusion peptide disclosed herein. In other embodiments, the agent is an antibody or aptamer that specifically binds SEQ ID NO:1.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A to 1C illustrate ER Proteostasis (FIG. 1A), Non-canonical ERAD (FIG. 1B), and GILZ and SGK1 (FIG. 1C).

FIG. 2 illustrates canonical ERAD. When proteins in the ER misfold (steps 1 and 2) they become toxic and must be degraded. Misfolded proteins are translocated across the ER membrane (3), then ubiquitylated by ER transmembrane E3 Ub ligases, such as Hrd1 (4), targeting them for degradation by proteasomes located on the cytosolic face of the ER (5).

FIG. 3 illustrates roles for SGK1 and GILZ in renal epithelial cells and cardiac myocytes.

FIGS. 4A to 4D show SGK1 and GILZ are induced in human HF and in mouse TAC. FIG. 4A shows SGK1 and GILZ mRNA levels assessed in human control (n=6) or hypertrophic HF (n=6). FIG. 4b shows SGK1 and GILZ mRNA levels assessed in mouse sham or TAC surgery heart samples 6W after the surgery. In FIGS. 4A and 4B, *≤p0.05 HF or TAC different from Con. FIGS. 4C and 4D show the same human and mouse heart samples analyzed in FIGS. 4A and 4B examined by immunoblotting for SGK1 and the direct substrates of SGK1, NDRG1 and NEDD4-2.

FIGS. 5A to 5E show SGK1 is induced during and required for NRVM growth. NRVMs were treated±PE for 48 h. The effect of PE on the following were measured.

FIG. 5A shows cell area and ANP mRNA. FIGS. 5B to 5E show SGK1 mRNA±siRNA to SGK1 (FIG. 5B), ANP mRNA±siRNA to SGK1 (FIG. 5C), Cell area±AdV-Con, SGK1-WT, -CA (FIG. 5D), ANP mRNA±AdV-Con, SGK1-WT, -CA (FIG. 5E). * and #≤p0.05 different from all other values.

FIGS. 6A to 6C show SGK1 is rapidly degraded by non-canonical ERAD in cardiac myocytes. FIGS. 6A to 6C show FLAG-SGK1WT (FIG. 6A), Δ(60) (FIG. 6B), or K6R (FIG. 6C) expressed in NRVM. ICF of FLAG and α-actinin. CHX was added for the times shown; FLAG IBs measured FLAG-SGK1 remaining, an indicator of the relative rates of degradation of each form of SGK1.

FIGS. 7A and 7B show relationship between SGK1 degradation rate and cardiac myocyte growth. FIGS. 7A and 7B show NRVM±AdV-Con, WT-SGK1, Δ(60) or K6R, ±PE for 48 h cell size (FIG. 7A), or ANP mRNA (FIG. 7B). *@#p<0.05 different from all other values by ANOVA.

FIGS. 8A to 8C show GILZ is induced and required for NRVM growth. FIGS. 8A to 8C show NRVMs±PE 48 h GILZ mRNA±GILZ siRNA (FIG. 8A), NRVM size±GILZ siRNA, AdV-SGK1-WT or KD (FIG. 8B), NRVM size±SGK1 siRNA±AdV-GILZ (FIG. 8C). *#@p<0.05 diff from other values (ANOVA).

FIGS. 9A to 9F show GILZ knockdown accelerates SGK1 degradation and decreases SGK1-mediated growth signaling. FIGS. 9A and 9B show hypothetical blocking of ER-targeting sequence on SGK1 by GILZ (FIG. 9A) and increased targeting of SGK1 to the ER and degradation upon GILZ knockdown (FIG. 9B). FIGS. 9C and 9D show siRNA control (FIG. 9C) or GILZ (FIG. 9D) used to knockdown GILZ in NRVM, then a CHX chase experiment was done to assess SGK1 degradation rates. FIGS. 9E and 9F show NRVM treated±GILZ siRNA±PE 48 h and SGK1 signaling to P-NEDD4-2 and P-S6K examined along with total levels of each and GILZ levels by immunoblotting.

FIGS. 10A to 10C show ectopic expression of GILZ slows SGK1 degradation.

FIG. 10A shows NRVM infected with various multiplicities of infection (MOI) of AdV-Con or AdV-GILZ then IB for FLAG, GILZ or GAPDH (n=3 cultures/treatment). FIG. 10B shows NRVMs infected with AdV-FLAG-SGK1-WT±AdV-Con or AdV-GILZ, then treated with CHX for the times shown then IB'd. FIG. 10C shows NRVMs infected±AdV-Con or AdV-GILZ, then threated±PE 48 h and SGK1 signaling to P-NEDD4-2 and P-S6K, GILZ and GAPDH were examined by IB. Note that ectopic GILZ increases upon PE treatment because the CMV promoter driving GILZ is induced by growth factors like PE.

FIGS. 11A to 11C show SGK1(122-157) Interrupts SGK1/GILZ Interaction and Decreases Cardiac Myocyte Growth. FIG. 11A illustrates how a small SGK1-related peptide could disrupt SGK1-GILZ interaction, increase SGK1 degradation and reduce cardiac myocyte growth. FIG. 11B shows SGK1(122-157) binds to GILZ. NRVM were infected with AdV-Con or FLAG-SGK-(122-157). IBs of cell extracts (CE) demonstrated appropriate SGK1 and FLAG expression (IBs 1-4). FLAG IP efficiently pulled down the FLAG-SGK peptide (5, 6) as well as GILZ (7, 8). FIG. 11C shows NRVM infected±AdV-SGK1 peptide, treated±PE, then assessed for ANP mRNA. *p<0.05 diff from Con t-test.

FIGS. 12A to 12E show TAT-SGK1-(Pep) Stops Growth of Myocytes. FIG. 12A shows AMVMs treated 24 h with PE±FITC-labeled TAT-SGK1-(Pep). FIG. 12B shows AMVMs treated 24 h±PE and the doses of TAT-SGK1-(Pep) shown then analyzed for hypertrophic growth, i.e. width to length ratio. FIG. 12C shows AMVMs ANP mRNA. FIG. 12D shows rate of endogenous SGK1 degradation in AMVMs±TAT-SGK1-(Pep). *, #, $<p 0.05 different from all other values ANOVA.

FIGS. 13A to 13C show effects of TAT-SGK1-(Pep) in AMVFs: TAT-SGK1-(Pep) [P] added to AMVFs at the conc shown±TGFβ. 48 h later cultures analyzed for α-SMA (FIG. 13A), periostin (FIG. 13B), IL1b (FIG. 13C) mRNAs shown by qRT-PCR. n=3 cultures/treatment*$ p<0.05 different from all other values (ANOVA).

FIGS. 14A to 14D show hypertrophy and functional decline are blunted in SGK1 cKO Mouse Hearts. FIG. 14A shows SGK1 IB of SGK1fl/fl mice treated with AAV9-Con (n=4) or AAV9-CRE (n=4) to generate SGK1 cKO mice. FIGS. 14B to 14E show ejection fraction (EF) by echo (FIG. 14B), HW/BW and/TL (FIG. 14C), LW/BW and LW/TL (FIG. 14D). *<p0.05 SGK1 cKO different from Con by t-test.

FIGS. 15A to 15E show generation of AAV9 for Ectopic Expression of SGK1 in mouse hearts. AAV9-FLAG-SGK1-WT, Δ(60), constitutively active (CA) and kinase dead (KD), GILZ were injected n=2 mice each with 1×10¹¹ or 3×10¹¹ viral particles of AAV9-Con (does not encode protein). After 3W, IB'd for FLAG, GILZ and GAPDH. Echo for LV mass (FIG. 15D) and EF (FIG. 15E) for AAV9-Con, WT and KD (n=4 to 8 as shown). *<p0.05 SGK1 cKO different from other values by ANOVA.

FIG. 16 shows immortalized cancer cells (HeLa) seeded at 250 cells/well on a 6-well plastic culture dish and treated with a scrambled peptide or the SGK1 peptide at concentrations of 0.1 μM, 1 μM, or 10 μM at timepoint 0 Hr. Cells were lifted off the dish and counted via hemocytomer after 24 Hr, 48 Hr, or 96 Hr in culture without refeeding during the course of the experiment.

FIG. 17 shows experimental design for in vivo evaluation of SGK1 peptide. Animals were randomly assigned to cohorts and a five-digit identifier number. All experimentalists were blinded to animal ID and group assignments until all data was compiled at which point the groups are revealed.

FIGS. 18A to 18D show LV mass (FIG. 18A), ejection fraction (FIG. 18B), cardiac stiffening (E/e′) (FIG. 18C), and A wave (FIG. 18D) in mice treated with control peptide, SGK1 peptide, or delayed treatment of SGK1 peptide. The mice were 4-weeks into a 6-week heart failure paradigm induced by transaortic constriction (TAC), a gold standard preclinical model of heart failure with reduced ejection fraction. Chronic administration of the SGK1 peptide shows no signs of untoward toxic effects and is conferring protection against TAC-induced left ventricular hypertrophy (LV Mass), systolic dysfunction (ejection fraction), diastolic dysfunction and cardiac stiffening (E/e′), and an early indicator of congestion (mitral atrial flow velocity). Furthermore, delayed administration of the SGK1 peptide starting at 2-weeks post-TAC shows signs of cardioprotection and reversal of early cardiac dysfunction and remodeling. Consistent pressure gradients to confirm standardized severity of the TAC procedure across cohorts is performed 1-week post-TAC via carotid flow Doppler ratios of the inominate to left common carotids.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

“Active”, with respect to a SGK1 polypeptide, refers to those forms, fragments, or domains of a SGK1 polypeptide which retain the biological activity of a SGK1 polypeptide.

“Naturally occurring SGK1 polypeptide” refers to a polypeptide produced by cells which have not been genetically engineered and specifically contemplates various polypeptides arising from post-translational modifications of the polypeptide including but not limited to acetylation, carboxylation, glycosylation, phosphorylation, lipidation and acylation.

“Conservative amino acid substitutions” result from replacing one amino acid with another having similar structural and/or chemical properties, such as the replacement of a leucine with an isoleucine or valine, an aspartate with a glutamate, or a threonine with a serine.

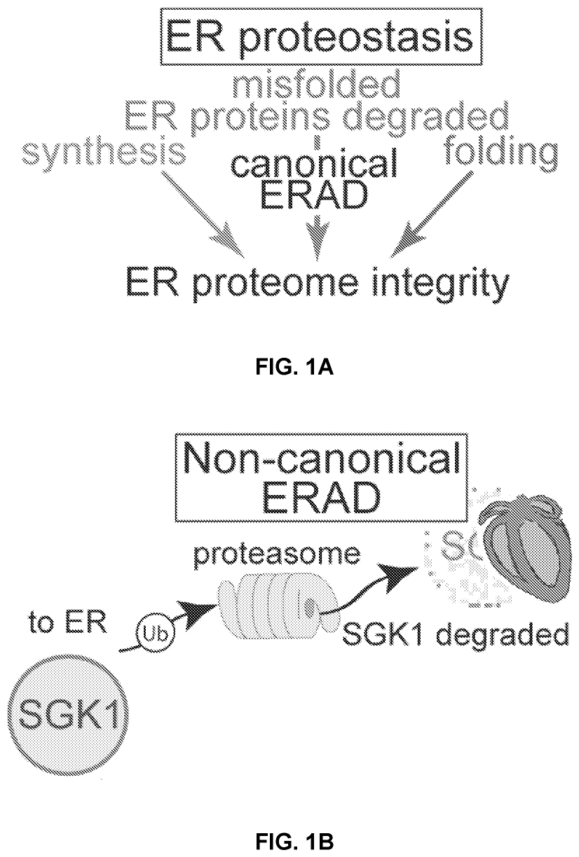

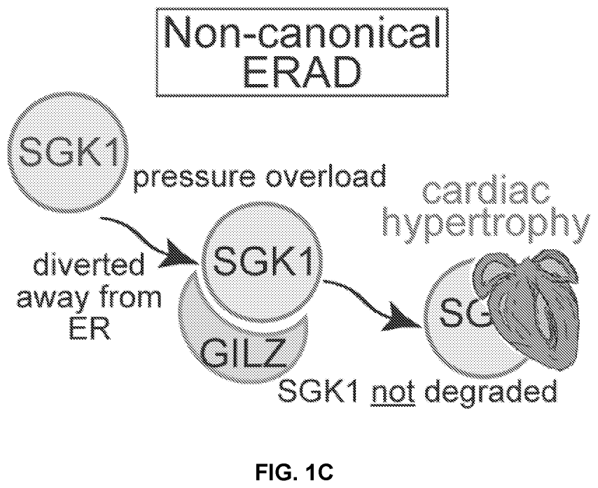

Many proteins required for heart function are made in the ER of cardiac myocytes, including calcium-handling proteins, receptors, and secreted proteins (Blackwood E A, et al. Cells. 2020 9). Thus, the ER in cardiac myocytes is an important site of protein homeostasis (proteostasis). ER proteostasis, including ER stress and the unfolded protein response, balances protein synthesis, folding and degradation of toxic ER misfolded proteins by ER associated degradation (ERAD) to ensure proteome integrity (FIG. 1A). The disclosed data support a new, non-canonical role for ERAD in the conditional degradation of the cytosolic, growth-promoting kinase, serum glucocorticoid kinase 1 (SGK1).

Canonical ERAD involves the ubiquitylation of misfolded ER proteins by ER transmembrane E3 ubiquitin ligases, such as Hrd1 (Sun Z and Brodsky J L. J Cell Biol. 2019). Since the catalytic domain of Hrd1 is on the cytosolic side of the ER, misfolded ER proteins must relocate out of the ER to be ubiquitylated by Hrd1, then degraded by cytosolic proteasomes. Increasing Hrd1 in cardiac myocytes decreases pathological cardiac hypertrophy in mice (Doroudgar S, et al. Circulation Research. 2015 117:536-546), leading to our concept that there is a mechanistic linkage between ERAD and growth of the heart that has not been studied; this link could involve SGK1, a regulator of Na reabsorption that has been studied in the kidney (Pearce D and Kleyman T R. J Clin Invest. 2007 117:592-5; Di Cristofano A. Curr Top Dev Biol. 2017 123:49-71), where it enhances Na reabsorption by increasing levels of epithelial cell Na channels (ENaCs) (Lang F, et al. Mol Membr Biol. 2014 31:29-36). When plasma Na is sufficient, SGK1 localizes to the ER of renal epithelial cells, and even though it is not misfolded, and is not an ER protein, SGK1 is ubiquitylated by Hrd1, then degraded by cytosolic proteasomes (Arteaga M F, et al. Proc Natl Acad Sci USA. 2006 103:11178-83) (FIG. 1B). When plasma Na is low, SGK1 is diverted from the ER by aldosterone- and glucocorticoid-inducible leucine zipper (GILZ) protein, which binds to, and masks the ER-targeting sequence of SGK1, protecting SGK1 from degradation (Soundararajan R, et al. J Biol Chem. 2010 285:39905-13) (FIG. 1C). In terms of growth, SGK1 has been extensively studied as a growth-promoter of cancer cells (Bruhn M A, et al. Growth Factors. 2010 28:394-408); however, other than a study in cultured cardiac myocytes, where activated SGK1 increased growth (Aoyama T, et al. Circulation. 2005 111:1652-9), roles for SGK1 and growth have not been well studied in the heart, in vivo, although activated SGK1 is arrhythmogenic in mouse hearts (Das S, et al. Circulation. 2012 126:2208-19).

SGK1 and GILZ were shown herein to be increased in pathological hypertrophic human and mouse hearts. In mice, SGK1 degradation was slowed during pressure overload. Cardiac-specific deletion of SGK1 in mice decreased pressure overload-induced hypertrophy, whereas overexpression of SGK1 increased it. Removing the SGK1 ER-targeting sequence, or overexpressing GILZ decreased SGK1 degradation in neonatal rat ventricular myocytes (NRVMs) and increased growth; GILZ knockdown decreased growth. A 35 amino acid region in SGK1 that interacts with GILZ was identified; ectopic expression of this peptide increased SGK1 degradation and decreased NRVM growth.

SGK1 is therefore a major inducer of pressure overload-induced cardiac pathology. During pressure overload, SGK1 levels, and thus, SGK1-mediated cardiac hypertrophy and subsequent pathology, are increased by GILZ-dependent diversion of SGK1 away from the ER, which decreases SGK1 degradation by non-canonical ERAD (FIG. 1B, 1C). Ectopic expression of an SGK1 peptide disrupts the GILZ-SGK1 interaction, increases SGK1 degradation, thus decreasing SGK1-mediated cardiac hypertrophy and subsequent pathology.

### Fusion Peptide

Disclosed herein is a fusion peptide comprising an inactive peptide fragment of Serum/Glucocorticoid Regulated Kinase 1 (SGK1) and a cell internalization sequence, wherein the SGK1 fragment is capable of binding and sequestering glucocorticoid-induced leucine zipper (GILZ).

### SGK1

Disclosed herein is a fusion peptide containing an inactive peptide fragment of SGK1 and an internalization sequence, optionally separated by a linker. In some embodiments, the inactive peptide fragment of SGK1 binds and sequesters GILZ, preventing it from binding and extending the half-life of endogenous SGK1.

The amino acid sequence for human SGK1 has the amino acid sequence:

(SEQ ID NO: 2)
MTVKTEAAKGTLTYSRMRGMVAILIAFMKQRRMGLNDFIQKIANNSYAC
KHPEVQSILKISQPQEPELMNANPSPPPSPSQQINLGPSSNPHAKPSDF
HFLKVIGKGSFGKVLLARHKAEEVFYAVKVLQKKAILKKKEEKHIMSER
NVLLKNVKHPFLVGLHFSFQTADKLYFVLDYINGGELFYHLQRERCFLE
PRARFYAAEIASALGYLHSLNIVYRDLKPENILLDSQGHIVLTDFGLCK
ENIEHNSTTSTFCGTPEYLAPEVLHKQPYDRTVDWWCLGAVLYEMLYGL
PPFYSRNTAEMYDNILNKPLQLKPNITNSARHLLEGLLQKDRTKRLGAK
DDFMEIKSHVFFSLINWDDLINKKITPPFNPNVSGPNDLRHFDPEFTEE
PVPNSIGKSPDSVLVTASVKEAAEAFLGFSYAPPTDSFL.

In some embodiments, the peptide fragment of SGK1 comprises at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 consecutive amino acids of VFYAVKVLQKKAILKKKEEKHIMSERNVLLKNVKHP (SEQ ID NO:1; aa 122-157 of SEQ ID NO:2), or a variant thereof having at least 85%, 86%, 87%, 88%, 89%, 90%, 91%, 92%, 93%, 94%, 95%, 96%, 97%, 98%, or 99% sequence identity to SEQ ID NO:1.

Therefore, in some embodiments, the peptide fragment of SGK1 comprises the amino acid sequence

(SEQ ID NO: 21)
FYAVKVLQKKAILKKKEEKHIMSERNVLLKNVKHP,
(SEQ ID NO: 22)
YAVKVLQKKAILKKKEEKHIMSERNVLLKNVKHP,
(SEQ ID NO: 23)
AVKVLQKKAILKKKEEKHIMSERNVLLKNVKHP,
(SEQ ID NO: 24)
VKVLQKKAILKKKEEKHIMSERNVLLKNVKHP,
(SEQ ID NO: 25)
KVLQKKAILKKKEEKHIMSERNVLLKNVKHP,
(SEQ ID NO: 26)
VLQKKAILKKKEEKHIMSERNVLLKNVKHP,
(SEQ ID NO: 27)
LQKKAILKKKEEKHIMSERNVLLKNVKHP,
(SEQ ID NO: 28)
QKKAILKKKEEKHIMSERNVLLKNVKHP,
(SEQ ID NO: 29)
KKAILKKKEEKHIMSERNVLLKNVKHP,
(SEQ ID NO: 30)
FYAVKVLQKKAILKKKEEKHIMSERNVLLKNVKH,
(SEQ ID NO: 31)
FYAVKVLQKKAILKKKEEKHIMSERNVLLKNVK,
(SEQ ID NO: 32)
FYAVKVLQKKAILKKKEEKHIMSERNVLLKNV,
(SEQ ID NO: 33)
FYAVKVLQKKAILKKKEEKHIMSERNVLLKN,
(SEQ ID NO: 34)
FYAVKVLQKKAILKKKEEKHIMSERNVLLK,
(SEQ ID NO: 35)
FYAVKVLQKKAILKKKEEKHIMSERNVLL,
(SEQ ID NO: 36)
FYAVKVLQKKAILKKKEEKHIMSERNVL,
(SEQ ID NO: 37)
FYAVKVLQKKAILKKKEEKHIMSERNV,
(SEQ ID NO: 38)
FYAVKVLQKKAILKKKEEKHIMSERN,
(SEQ ID NO: 39)
YAVKVLQKKAILKKKEEKHIMSERNVLLKNVKH,
(SEQ ID NO: 40)
YAVKVLQKKAILKKKEEKHIMSERNVLLKNVK,
(SEQ ID NO: 41)
YAVKVLQKKAILKKKEEKHIMSERNVLLKNV,
(SEQ ID NO: 42)
YAVKVLQKKAILKKKEEKHIMSERNVLLKN,
(SEQ ID NO: 43)
YAVKVLQKKAILKKKEEKHIMSERNVLLK,
(SEQ ID NO: 44)
YAVKVLQKKAILKKKEEKHIMSERNVLL,
(SEQ ID NO: 45)
YAVKVLQKKAILKKKEEKHIMSERNVL,
(SEQ ID NO: 46)
YAVKVLQKKAILKKKEEKHIMSERNV,
(SEQ ID NO: 47)
AVKVLQKKAILKKKEEKHIMSERNVLLKNVKH,
(SEQ ID NO: 48)
AVKVLQKKAILKKKEEKHIMSERNVLLKNVK,
(SEQ ID NO: 49)
AVKVLQKKAILKKKEEKHIMSERNVLLKNV,
(SEQ ID NO: 50)
AVKVLQKKAILKKKEEKHIMSERNVLLKN,
(SEQ ID NO: 51)
AVKVLQKKAILKKKEEKHIMSERNVLLK,
(SEQ ID NO: 52)
AVKVLQKKAILKKKEEKHIMSERNVLL,
(SEQ ID NO: 53)
AVKVLQKKAILKKKEEKHIMSERNVL,
(SEQ ID NO: 54)
VKVLQKKAILKKKEEKHIMSERNVLLKNVKH,
(SEQ ID NO: 55)
VKVLQKKAILKKKEEKHIMSERNVLLKNVK,
(SEQ ID NO: 56)
VKVLQKKAILKKKEEKHIMSERNVLLKNV,
(SEQ ID NO: 57)
VKVLQKKAILKKKEEKHIMSERNVLLKN,
(SEQ ID NO: 58)
VKVLQKKAILKKKEEKHIMSERNVLLK,
(SEQ ID NO: 59)
VKVLQKKAILKKKEEKHIMSERNVLL,
(SEQ ID NO: 60)
KVLQKKAILKKKEEKHIMSERNVLLKNVKH,
(SEQ ID NO: 61)
KVLQKKAILKKKEEKHIMSERNVLLKNVK,
(SEQ ID NO: 62)
KVLQKKAILKKKEEKHIMSERNVLLKNV,
(SEQ ID NO: 63)
KVLQKKAILKKKEEKHIMSERNVLLKN,
(SEQ ID NO: 64)
KVLQKKAILKKKEEKHIMSERNVLLK,
(SEQ ID NO: 65)
VLQKKAILKKKEEKHIMSERNVLLKNVKH,
(SEQ ID NO: 66)
VLQKKAILKKKEEKHIMSERNVLLKNVK,
(SEQ ID NO: 67)
VLQKKAILKKKEEKHIMSERNVLLKNV,
(SEQ ID NO: 68)
VLQKKAILKKKEEKHIMSERNVLLKN,
(SEQ ID NO: 69)
LQKKAILKKKEEKHIMSERNVLLKNVKH,
(SEQ ID NO: 70)
LQKKAILKKKEEKHIMSERNVLLKNVK,
(SEQ ID NO: 71)
LQKKAILKKKEEKHIMSERNVLLKNV,
(SEQ ID NO: 72)
QKKAILKKKEEKHIMSERNVLLKNVKH,
(SEQ ID NO: 73)
QKKAILKKKEEKHIMSERNVLLKNVK,
or
(SEQ ID NO: 74)
KKAILKKKEEKHIMSERNVLLKNVKH,

or a variant thereof having at least 85%, 86%, 87%, 88%, 89%, 90%, 91%, 92%, 93%, 94%, 95%, 96%, 97%, 98%, or 99% sequence identity to any one of SEQ ID NOs:21 to 74.

In some embodiments, the peptide fragment of SGK1 amino acids 1-97 of SEQ ID NO:2.

Therefore, in some embodiments, the peptide fragment of SGK1 contains less than 37, 38, 39, 40, 41, 42, 43, 44, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, or 400 consecutive amino acids from SEQ ID NO:2.

In some embodiments, the fusion peptide contains 2 or more SGK1 fragments, including 2, 3, 4, 5, 6, 7, 8, 9, 10 or more SGK1 fragments. These fragments can in some embodiments, be separated by a linker.

### Internalization Sequences

The provided polypeptide can further constitute a fusion protein or otherwise have additional N-terminal, C-terminal, or intermediate amino acid sequences, e.g., linkers or tags. “Linker”, as used herein, is an amino acid sequences or insertion that can be used to connect or separate two distinct polypeptides or polypeptide fragments, wherein the linker does not otherwise contribute to the essential function of the composition. A polypeptide provided herein, can have an amino acid linker comprising, for example, the amino acids GLS, ALS, or LLA. A “tag”, as used herein, refers to a distinct amino acid sequence that can be used to detect or purify the provided polypeptide, wherein the tag does not otherwise contribute to the essential function of the composition. The provided polypeptide can further have deleted N-terminal, C-terminal or intermediate amino acids that do not contribute to the essential activity of the polypeptide.

The disclosed composition can be linked to an internalization sequence or a protein transduction domain to effectively enter the cell. Recent studies have identified several cell penetrating peptides, including the TAT transactivation domain of the HIV virus, antennapedia, and transportan that can readily transport molecules and small peptides across the plasma membrane (Schwarze et al., Science. 1999 285(5433):1569-72; Derossi et al. J Biol Chem. 1996 271(30):18188-93; Yuan et al., Cancer Res. 2002 62(15):4186-90). More recently, polyarginine has shown an even greater efficiency of transporting peptides and proteins across the plasma, membrane making it an attractive tool for peptide mediated transport (Fuchs and Raines, Biochemistry. 2004 43(9):2438-44). Nona-arginine has been described as one of the most efficient polyarginine based protein transduction domains, with maximal uptake of significantly greater than TAT or antennapeadia. Peptide mediated cytotoxicity has also been shown to be less with polyarginine-based internalization sequences. R₉ mediated membrane transport is facilitated through heparan sulfate proteoglycan binding and endocytic packaging. Once internalized, heparan is degraded by heparanases, releasing R₉ which leaks into the cytoplasm (Deshayes et al., Cell Mol Life Sci. 2005 62(16):1839-49). Studies have recently shown that derivatives of polyarginine can deliver a full length p53 protein to oral cancer cells, suppressing their growth and metastasis, defining polyarginine as a potent cell penetrating peptide (Takenobu et al., Mol Cancer Ther. 2002 1(12):1043-9).

Thus, the provided polypeptide can comprise a cellular internalization transporter or sequence. The cellular internalization sequence can be any internalization sequence known or newly discovered in the art, or conservative variants thereof. Non-limiting examples of cellular internalization transporters and sequences include Polyarginine (e.g., R₉), Antennapedia sequences, TAT, HIV-Tat, Penetratin, Antp-3A (Antp mutant), Buforin II, Transportan, MAP (model amphipathic peptide), K-FGF, Ku70, Prion, pVEC, Pep-1, SynB1, Pep-7, HN-1, BGSC (Bis-Guanidinium-Spermidine-Cholesterol, and BGTC (Bis-Guanidinium-Tren-Cholesterol) (see Table 1).

TABLE 1
Cell Internalization Transporters

| Name | Sequence | SEQ ID NO |
|---|---|---|
| Polyarginine | RRRRRRRRR | SEQ ID NO: 3 |
| Antp | RQPKIWFPNRRKPWKK | SEQ ID NO: 4 |
| HIV-Tat | GRKKRRQRPPQ | SEQ ID NO: 5 |
| Penetratin | RQIKIWFQNRRMKWKK | SEQ ID NO: 6 |
| Antp-3A | RQIAIWFQNRRMKWAA | SEQ ID NO: 7 |
| Tat | RKKRRQRRR | SEQ ID NO: 8 |
| Buforin II | TRSSRAGLQFPVGRVHRLLRK | SEQ ID NO: 9 |
| Transportan | GWTLNSAGYLLGKINKALAALAKKIL | SEQ ID NO: 10 |
| model amphipathic | KLALKLALKALKAALKLA | SEQ ID NO: 11 |
| peptide (MAP) | | |
| K-FGF | AAVALLPAVLLALLAP | SEQ ID NO: 12 |
| Ku70 | VPMLKPMLKE | SEQ ID NO: 13 |
| Prion | MANLGYWLLALFVTMWTDVGLCKK | SEQ ID NO: 14 |
| | RPKP | |
| pVEC | LLIILRRRIRKQAHAHSK | SEQ ID NO: 15 |
| Pep-1 | KETWWETWWTEWSQPKKKRKV | SEQ ID NO: 16 |
| SynB1 | RGGRLSYSRRRFSTSTGR | SEQ ID NO: 17 |
| Pep-7 | SDLWEMMMVSLACQY | SEQ ID NO: 18 |
| HN-1 | TSPLNIHNGQKL | SEQ ID NO: 19 |
| BGSC (Bis- Guanidinium- Spermidine- Cholesterol) | 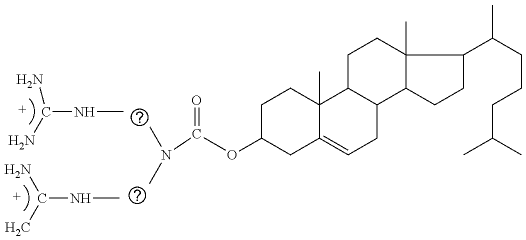 | |
| | BGSC | |
| BGTC (Bis- Guanidinium-Tren- Cholesterol) | 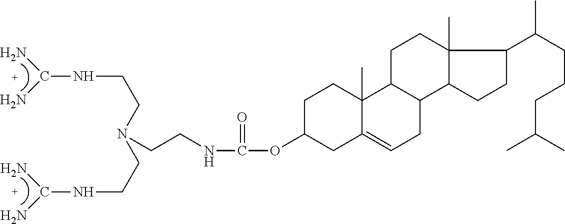 [SMILES: CC(C)CCCC(C)C1CCC2C3CC=C4CC(OC(=O)NCCN(CCNC(N)N)CCNC(N)N)CCC4(C)C3CCC12C] | |

8GTC

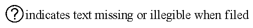

Therefore, in some embodiments, the fusion peptide comprises or consists of the amino acid sequence GRKKRRQRRRPQVFYAVKVLQKKAILKKKEEKHIMSERNVLLKNVKHP (SEQ ID NO:20). Therefore, in some embodiments, the fusion peptide comprises the amino acid sequence

Any other internalization sequences now known or later identified can be combined with a peptide of the invention.

### Linkers

Components of the fusion protein may be linked by a linking moiety such as a peptide linker. Preferably, the linker does not interfere significantly with the structure of each functional component within the fusion protein. In some embodiments, the linker moiety is a peptide linker. In some embodiments, the peptide linker comprises 2 to 100 amino acids. In some embodiments, the peptide linker comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 but no greater than 100 amino acids. In some embodiments, the peptide linker is between 5 to 75, 5 to 50, 5 to 25, 5 to 20, 5 to 15, 5 to 10 or 5 to 9 amino acids in length. Exemplary linkers include linear peptides having at least two amino acid residues such as Gly-Gly, Gly-Ala-Gly, Gly-Pro-Ala, Gly-Gly-Gly-Gly-Ser (SEQ ID NO:75). Suitable linear peptides include poly glycine, polyserine, polyproline, polyalanine and oligopeptides consisting of alanyl and/or serinyl and/or prolinyl and/or glycyl amino acid residues. In some embodiments, the peptide linker comprises the amino acid sequence selected from the group consisting of Gly₉ (SEQ ID NO:76), Glue (SEQ ID NO:77), Ser₉ (SEQ ID NO:78), Gly₅-Cys-Pro₂-Cys (SEQ ID NO:79), (Gly₄-Ser)₃ (SEQ ID NO:80), Ser-Cys-Val-Pro-Leu-Met-Arg-Cys-Gly-Gly-Cys-Cys-Asn (SEQ ID NO:81), Pro-Ser-Cys-Val-Pro-Leu-Met-Arg-Cys-Gly-Gly-Cys-Cys-Asn (SEQ ID NO:82), Gly-Asp-Leu-Ile-Tyr-Arg-Asn-Gln-Lys (SEQ ID NO:83), and Gly₉-Pro-Ser-Cys-Val-Pro-Leu-Met-Arg-Cys-Gly-Gly-Cys-Cys-Asn (SEQ ID NO:84).

Linker moieties can also be made from other polymers, such as polyethylene glycol. Such linkers can have from 10 to 1000, 10 to 500, 10 to 250, 10 to 100, or 10 to 50 ethylene glycol monomer units. Suitable polymers should be of a size similar to the size occupied by the appropriate range of amino acid residues. A typical sized polymer would provide a spacing of from about 10-25 angstroms.

The linker moiety may be a protein multivalent linker that has branched “arms” that link multiple fusion protein components in a non-linear fashion. In some embodiments, a multivalent linker has about 3 to 40 amino acid residues, all or some of which provide attachment sites for conjugation with fusion protein components. Alpha amino groups and alpha carboxylic acids can serve as attachment sites. Exemplary multivalent linkers include, but are not limited to, polylysines, polyornithines, polycysteines, polyglutamic acid and polyaspartic acid. Optionally, amino acid residues with inert side chains, e.g., glycine, alanine and valine, can be included in the amino acid sequence. The linkers may also be a non-peptide chemical entity such as a chemical linker that is suitable for administration (e.g., ocular administration) once attached to a fusion protein component. The chemical linker may be a bifunctional linker, each of which reacts with a fusion protein component. Alternatively, the chemical linker may be a branched linker that has a multiplicity of appropriately spaced reactive groups, each of which can react with a functional group of a fusion protein component. The fusion protein components are attached by way of reactive functional groups and are spaced such that steric hindrance does not substantially interfere with formation of covalent bonds between some of the reactive functional groups (e.g., amines, carboxylic acids, alcohols, aldehydes and thiols) and the peptide. Examples of linker moieties include, but are not limited to, those disclosed in Tarn, J. P., et al., J. of Immunol Methods, 1996, 196:17-32.

### Viral Vectors

Also provided herein are viral vectors comprising a nucleic acid encoding a fusion protein described herein. Viral vectors can be used for delivery of a nucleic acid encoding a fusion protein or fusion protein component for expression of the protein in a target cell within a particular target tissue (e.g., a diseased tissue). Many species of virus are known, and many have been studied for purposes of delivering nucleic acids to target cells. The exogenous nucleic acid can be inserted into a vector such as adenovirus, partially-deleted adenovirus, fully-deleted adenovirus, adeno-associated virus (AAV), retrovirus, lentivirus, and so forth for delivery to a cell. In some embodiments, the cell is in an individual and the virus is delivered via an intravenous, intramuscular, intraportal or other route of administration. The most commonly used viral vectors include those derived from adenoviruses, adeno-associated viruses (AAV) and retroviruses, including lentiviruses, such as human immunodeficiency virus (HIV). For exemplary viral vectors see U.S. Pat. No. 7,928,072 and WO2006/113277, both of which are incorporated herein by reference in their entirety.

In some embodiments, the viral vector is a recombinant AAV particle comprising a nucleic acid comprising one or two AAV ITRs and a sequence encoding a fusion protein described herein flanked by one or two ITRs. The nucleic acid is encapsidated in the AAV particle. The AAV particle also comprises capsid proteins. In some embodiments, the nucleic acid comprises operatively linked components in the direction of transcription, control sequences including transcription initiation and termination sequences, and the protein coding sequence(s) of interest (e.g., nucleic acid encoding a fusion protein). These components are flanked on the 5′ and 3′ end by functional AAV ITR sequences. By “functional AAV ITR sequences” it is meant that the ITR sequences function as intended for the rescue, replication and packaging of the AAV virion. See Davidson et al., PNAS, 2000, 97(7)3428-32; Passini et al., J. Virol., 2003, 77(12):7034-40; and Pechan et al., Gene Ther., 2009, 16:10-16, all of which are incorporated herein in their entirety by reference. For practicing some aspects of the invention, the recombinant vectors comprise at least all of the sequences of AAV essential for encapsidation and the physical structures for infection by the rAAV. AAV ITRs for use in the vectors of the invention need not have a wild-type nucleotide sequence (e.g., as described in Kotin, Hum. Gene Ther., 1994, 5:793-801), and may be altered by the insertion, deletion or substitution of nucleotides or the AAV ITRs may be derived from any of several AAV serotypes. More than 40 serotypes of AAV are currently known, and new serotypes and variants of existing serotypes continue to be identified. See Gao et al., PNAS, 2002, 99(18): 11854-6; Gao et al., PNAS, 2003, 100(10):6081-6; and Bossis et al., J. Virol., 2003, 77(12):6799-810. Use of any AAV serotype is considered within the scope of the present invention. In some embodiments, a rAAV vector is a vector derived from an AAV serotype, including without limitation, AAV1, AAV2, AAV3, AAV4, AAV5, AA6, AAV7, AAV8, AAV9, AAVrh.8, and AAVrh.10. In some embodiments, the nucleic acid in the AAV comprises an ITR of AAV1, AAV2, AAV3, AAV4, AAV5, AA6, AAV7, AAV8, AAV9, AAVrh.8, or AAVrh.10. In some embodiments, a nucleic acid encoding a fusion protein selected from the group consisting of SEQ ID NOs:12-15 is flanked by at least one AAV ITR. In some embodiments, the nucleic acid is selected from the group consisting of SEQ ID Nos:21-24. In further embodiments, the rAAV particle comprises capsid proteins of AAV1, AAV2, AAV3, AAV4, AAV5, AA6, AAV7, AAV8, AAV9, AAVrh.8, or AAVrh.10.

Different AAV serotypes are used to optimize transduction of particular target cells or to target specific cell types within a particular target tissue (e.g., a diseased tissue). A rAAV particle can comprise viral proteins and viral nucleic acids of the same serotype or a mixed serotype. For example, a rAAV particle can comprise AAV2 capsid proteins and at least one AAV2 ITR or it can comprise AAV2 capsid proteins and at least one AAV1 ITR. In another example, a rAAV particle can comprise AAV1 capsid proteins and at least one AAV2 ITR. In yet another example, a rAAV particle can comprise capsid proteins from both AAV1 and AAV2, and further comprise at least one AAV2 ITR. Any combination of AAV serotypes for production of a rAAV particle is provided herein as if each combination had been expressly stated herein.

The rAAV particles can be produced using methods know in the art. See, e.g., U.S. Pat. Nos. 6,566,118, 6,989,264, 6,995,006. In practicing the invention, host cells for producing rAAV particles include mammalian cells, insect cells, plant cells, microorganisms and yeast. Host cells can also be packaging cells in which the AAV rep and cap genes are stably maintained in the host cell or producer cells in which the AAV vector genome is stably maintained. Exemplary packaging and producer cells are derived from 293, A549 or HeLa cells. AAV vectors are purified and formulated using standard techniques known in the art.

In some aspects, a method is provided for producing any rAAV particle as disclosed herein comprising (a) culturing a host cell under a condition that rAAV particles are produced, wherein the host cell comprises (i) one or more AAV package genes, wherein each said AAV packaging gene encodes an AAV replication or encapsidation protein; (ii) an rAAV pro-vector comprising a nucleic acid encoding any fusion protein disclosed herein flanked by at least one AAV ITR, and (iii) an AAV helper function; and (b) recovering the rAAV particles produced by the host cell. In some embodiments, a nucleic acid encodes a fusion protein selected from the group consisting of SEQ ID NOs:12-15. In some embodiments, said at least one AAV ITR is selected from the group consisting of AAV1, AAV2, AAV3, AAV4, AAV5, AA6, AAV7, AAV8, AAV9, AAVrh.8, and AAVrh.10 ITR. In some embodiments, said encapsidation protein is selected from the group consisting of AAV1, AAV2, AAV3, AAV4, AAV5, AA6, AAV7, AAV8, AAV9, AAVrh.8, and AAVrh.10 capsid protein. In a further embodiment, the rAAV particles are purified. The term “purified” as used herein includes a preparation of rAAV particles devoid of at least some of the other components that may also be present where the rAAV particles naturally occur or are initially prepared from. Thus, for example, isolated rAAV particles may be prepared using a purification technique to enrich it from a source mixture, such as a culture lysate or production culture supernatant. Enrichment can be measured in a variety of ways, such as, for example, by the proportion of DNase-resistant particles (DRPs) present in a solution, or by infectivity, or it can be measured in relation to a second, potentially interfering substance present in the source mixture, such as contaminants, including production culture contaminants or in-process contaminants, including helper virus, media components, and the like.

Also provided herein are pharmaceutical compositions comprising a rAAV particle comprising a nucleic acid encoding a fusion protein disclosed herein and a pharmaceutically acceptable carrier. The pharmaceutical compositions may be suitable for a variety of modes of administration described herein, including for example systemic or localized administration. A pharmaceutical composition of a rAAV comprising a nucleic acid encoding a fusion protein described herein can be introduced systemically, e.g., by intravenous injection, by catheter, see U.S. Pat. No. 5,328,470, or by stereotactic injection, Chen et al., 1994, PNAS, 91: 3054-3057. The pharmaceutical compositions can be in the form of eye drops, injectable solutions, or in a form suitable for inhalation or oral administration. In some embodiments, the pharmaceutical compositions comprising a fusion protein described herein and a pharmaceutically acceptable carrier is suitable for administration to human. In some embodiments, the pharmaceutical compositions comprising a fusion protein described herein and a pharmaceutically acceptable carrier is suitable for intravitreal injection or topical application to the eye. Such pharmaceutically acceptable carriers can be sterile liquids, such as water and oil, including those of petroleum, animal, vegetable or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. The pharmaceutical composition may further comprise additional ingredients, for example preservatives, buffers, tonicity agents, antioxidants and stabilizers, nonionic wetting or clarifying agents, viscosity-increasing agents, and the like. The pharmaceutical compositions described herein can be packaged in single unit dosages or in multidosage forms. The compositions are generally formulated as sterile and substantially isotonic solution. Compositions can also be formulated to have osmotic values that are compatible with the aqueous humor of the eye and ophthalmic tissues. Such osmotic values will generally be in the range of from about 200 to about 400 mOsm/kg, but will preferably be about 300 mOsm/kg. Ophthalmic solutions useful for storing and/or delivering expression vectors or viral vectors have been disclosed, for example, in WO03077796A2.

### Pharmaceutical Compositions

Disclosed herein are pharmaceutical composition containing SGK1 peptide fragments disclosed herein in conjunction with a pharmaceutically acceptable carrier, for any of the therapeutic effects discussed above. The compositions may be administered alone or in combination with at least one other agent, such as a stabilizing compound, which may be administered in any sterile, biocompatible pharmaceutical carrier including, but not limited to, saline, buffered saline, dextrose, and water. The compositions may be administered to a patient alone, or in combination with other agents, drugs or hormones.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EMT™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyetheylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin. Sterile injectable solutions can be prepared by incorporating the active compound (e.g., a polypeptide or antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration. The instructions for administration can specify use of the composition for cardiovascular diseases, cancer, endocrinological diseases, metabolic diseases, inflammation, gastroenterological diseases, hematological diseases, respiratory diseases, neurological diseases and urological diseases.

### Methods of Treatment

SGK1 is expressed in various human tissues and is involved in a number of diseases and disorders that can be treated using the disclosed fusion peptides. For example, disclosed herein are prophylactic and therapeutic methods for cardiovascular diseases, cancer, endocrinological diseases, metabolic diseases, inflammation, gastroenterological diseases, hematological diseases, respiratory diseases, neurological diseases and urological diseases.

Therefore, disclosed herein is a method for treating a disease associated with aberrant SGK1 activity in a subject, comprising administering to the subject an agent that blocks the binding of endogenous SGK1 to endogenous GILZ.

In some embodiments, the agent is a fusion peptide containing an inactive peptide fragment of SGK1 and a cell internalization sequence as disclosed herein. In some embodiments, the agent is an antibody or aptamer that specifically binds SEQ ID NO:1.

### Neuroloqy

CNS disorders include disorders of the central nervous system as well as disorders of the peripheral nervous system. CNS disorders include, but are not limited to brain injuries, cerebrovascular diseases and their consequences, Parkinson's disease, corticobasal degeneration, motor neuron disease, dementia, including ALS, multiple sclerosis, traumatic brain injury, stroke, post-stroke, post-traumatic brain injury, and small-vessel cerebrovascular disease. Dementias, such as Alzheimer's disease, vascular dementia, dementia with Lewy bodies, frontotemporal dementia and Parkinsonism linked to chromosome 17, frontotemporal dementias, including Pick's disease, progressive nuclear palsy, corticobasal degeneration, Huntington's disease, thalamic degeneration, Creutzfeld-Jakob dementia, HIV dementia, schizophrenia with dementia, and Korsakoff's psychosis, within the meaning of the definition are also considered to be CNS disorders.

Similarly, cognitive-related disorders, such as mild cognitive impairment, age-associated memory impairment, age-related cognitive decline, vascular cognitive impairment, attention deficit disorders, attention deficit hyperactivity disorders, and memory disturbances in children with learning disabilities are also considered to be CNS disorders.

Pain, within the meaning of this definition, is also considered to be a CNS disorder. Pain can be associated with CNS disorders, such as multiple sclerosis, spinal cord injury, sciatica, failed back surgery syndrome, traumatic brain injury, epilepsy, Parkinson's disease, post-stroke, and vascular lesions in the brain and spinal cord (e.g., infarct, hemorrhage, vascular malformation). Non-central neuropathic pain includes that associated with post mastectomy pain, phantom feeling, reflex sympathetic dystrophy (RSD), trigeminal neuralgiaradioculopathy, post-surgical pain, HIV/AIDS related pain, cancer pain, metabolic neuropathies (e.g., diabetic neuropathy, vasculitic neuropathy secondary to connective tissue disease), paraneoplastic polyneuropathy associated, for example, with carcinoma of lung, or leukemia, or lymphoma, or carcinoma of prostate, colon or stomach, trigeminal neuralgia, cranial neuralgias, and post-herpetic neuralgia. Pain associated with peripheral nerve damage, central pain (i.e. due to cerebral ischemia) and various chronic pain i.e., lumbago, back pain (low back pain), inflammatory and/or rheumatic pain. Headache pain (for example, migraine with aura, migraine without aura, and other migraine disorders), episodic and chronic tension-type headache, tension-type like headache, cluster headache, and chronic paroxysmal hemicrania are also CNS disorders.

Visceral pain such as pancreatits, intestinal cystitis, dysmenorrhea, irritable Bowel syndrome, Crohn's disease, biliary colic, ureteral colic, myocardial infarction and pain syndromes of the pelvic cavity, e.g., vulvodynia, orchialgia, urethral syndrome and protatodynia are also CNS disorders.

Also considered to be a disorder of the nervous system are acute pain, for example postoperative pain, and pain after trauma.

The human SGK is highly expressed in the following brain tissues: brain, Alzheimer brain, cerebellum (right), cerebellum (left), cerebral cortex, Alzheimer cerebral cortex, frontal lobe, Alzheimer brain frontal lobe, occipital lobe, parietal lobe, temporal lobe, substantia nigra, corpus callosum, hippocampus, spinal cord, neuroblastoma SH-SY5Y cells. The expression in brain tissues and in particular the differential expression between diseased tissue Alzheimer brain and healthy tissue brain, between diseased tissue Alzheimer cerebral cortex and healthy tissue cerebral cortex, between diseased tissue Alzheimer brain frontal lobe and healthy tissue frontal lobe demonstrates that the human SGK or mRNA can be utilized to diagnose nervous system diseases. Additionally the activity of the human SGK can be modulated to treat nervous system diseases.

### Cardiovascular Disorders

Heart failure is defined as a pathophysiological state in which an abnormality of cardiac function is responsible for the failure of the heart to pump blood at a rate commensurate with the requirement of the metabolizing tissue. It includes all forms of pumping failures such as high-output and low-output, acute and chronic, right-sided or left-sided, systolic or diastolic, independent of the underlying cause.

Myocardial infarction (MI) is generally caused by an abrupt decrease in coronary blood flow that follows a thrombotic occlusion of a coronary artery previously narrowed by arteriosclerosis. MI prophylaxis (primary and secondary prevention) is included as well as the acute treatment of MI and the prevention of complications.

Ischemic diseases are conditions in which the coronary flow is restricted resulting in a perfusion which is inadequate to meet the myocardial requirement for oxygen. This group of diseases includes stable angina, unstable angina and asymptomatic ischemia.

Arrhythmias include all forms of atrial and ventricular tachyarrhythmias, atrial tachycardia, atrial flutter, atrial fibrillation, atrio-ventricular reentrant tachycardia, pre-excitation syndrome, ventricular tachycardia, ventricular flutter, ventricular fibrillation, as well as bradycardic forms of arrhythmias.

Hypertensive vascular diseases include primary as well as all kinds of secondary arterial hypertension, renal, endocrine, neurogenic, others. The genes may be used as drug targets for the treatment of hypertension as well as for the prevention of all complications arising from cardiovascular diseases.

Peripheral vascular diseases are defined as vascular diseases in which arterial and/or venous flow is reduced resulting in an imbalance between blood supply and tissue oxygen demand. It includes chronic peripheral arterial occlusive disease (PAOD), acute arterial thrombosis and embolism, inflammatory vascular disorders, Raynaud's phenomenon and venous disorders.

Atherosclerosis is a cardiovascular disease in which the vessel wall is remodeled, compromising the lumen of the vessel. The atherosclerotic remodeling process involves accumulation of cells, both smooth muscle cells and monocyte/macrophage inflammatory cells, in the intima of the vessel wall. These cells take up lipid, likely from the circulation, to form a mature atherosclerotic lesion. Although the formation of these lesions is a chronic process, occurring over decades of an adult human life, the majority of the morbidity associated with atherosclerosis occurs when a lesion ruptures, releasing thrombogenic debris that rapidly occludes the artery. When such an acute event occurs in the coronary artery, myocardial infarction can ensue, and in the worst case, can result in death.

The formation of the atherosclerotic lesion can be considered to occur in five overlapping stages such as migration, lipid accumulation, recruitment of inflammatory cells, proliferation of vascular smooth muscle cells, and extracellular matrix deposition. Each of these processes can be shown to occur in man and in animal models of atherosclerosis, but the relative contribution of each to the pathology and clinical significance of the lesion is unclear.

Thus, a need exists for therapeutic methods and agents to treat cardiovascular pathologies, such as atherosclerosis and other conditions related to coronary artery disease.

Cardiovascular diseases include but are not limited to disorders of the heart and the vascular system like congestive heart failure, myocardial infarction, ischemic diseases of the heart, all kinds of atrial and ventricular arrhythmias, hypertensive vascular diseases, peripheral vascular diseases, and atherosclerosis.

Too high or too low levels of fats in the bloodstream, especially cholesterol, can cause long-term problems. The risk to develop atherosclerosis and coronary artery or carotid artery disease (and thus the risk of having a heart attack or stroke) increases with the total cholesterol level increasing. Nevertheless, extremely low cholesterol levels may not be healthy. Examples of disorders of lipid metabolism are hyperlipidemia (abnormally high levels of fats (cholesterol, triglycerides, or both) in the blood, may be caused by family history of hyperlipidemia), obesity, a high-fat diet, lack of exercise, moderate to high alcohol consumption, cigarette smoking, poorly controlled diabetes, and an underactive thyroid gland), hereditary hyperlipidemias (type I hyperlipoproteinemia (familial hyperchylomicronemia), type II hyperlipoproteinemia (familial hypercholesterolemia), type Ill hyperlipoproteinemia, type IV hyperlipoproteinemia, or type V hyperlipoproteinemia), hypolipoproteinemia, lipidoses (caused by abnormalities in the enzymes that metabolize fats), Gaucher's disease, Niemann-Pick disease, Fabry's disease, Wolman's disease, cerebrotendinous xanthomatosis, sitosterolemia, Refsum's disease, or Tay-Sachs disease.

Kidney disorders may lead to hypertension or hypotension. Examples for kidney problems possibly leading to hypertension are renal artery stenosis, pyelonephritis, glomerulonephritis, kidney tumors, polycystic kidney disease, injury to the kidney, or radiation therapy affecting the kidney. Excessive urination may lead to hypotension.

The human SGK is highly expressed in the following cardiovascular related tissues: fetal heart, heart, pericardium, heart atrium (right), heart atrium (left), heart apex, Purkinje fibers, interventricular septum, coronary artery smooth muscle primary cells, HUVEC cells, adrenal gland, liver, liver tumor, fetal kidney, kidney, kidney tumor. Expression in the above mentioned tissues demonstrates that the human SGK or mRNA can be utilized to diagnose of cardiovascular diseases. Additionally the activity of the human SGK can be modulated to treat cardiovascular diseases.

The human SGK is highly expressed in liver tissues: liver, liver tumor. Expression in liver tissues demonstrates that the human SGK or mRNA can be utilized to diagnose of dyslipidemia disorders as a cardiovascular disorder. Additionally the activity of the human SGK can be modulated to treat—but not limited to—dyslipidemia disorders.

The human SGK is highly expressed in kidney tissues: fetal kidney, kidney, kidney tumor. Expression in kidney tissues demonstrates that the human SGK or mRNA can be utilized to diagnose of blood pressure disorders as a cardiovascular disorder. Additionally the activity of the human SGK can be modulated to treat—but not limited to—blood pressure disorders as hypertension or hypotension.

The human SGK is highly expressed in adrenal gland. Expression in adrenal gland tissues demonstrates that the human SGK or mRNA can be utilized to diagnose of blood pressure disorders as an cardiovascular disorder. Additionally the activity of the human SGK can be modulated to treat—but not limited to—blood pressure disorders as hypertension or hypotension.

### Hematological Disorders

Hematological disorders comprise diseases of the blood and all its constituents as well as diseases of organs and tissues involved in the generation or degradation of all the constituents of the blood. They include but are not limited to 1) Anemias, 2) Myeloproliferative Disorders, 3) Hemorrhagic Disorders, 4) Leukopenia, 5) Eosinophilic Disorders, 6) Leukemias, 7) Lymphomas, 8) Plasma Cell Dyscrasias, 9) Disorders of the Spleen in the course of hematological disorders. Disorders according to 1) include, but are not limited to anemias due to defective or deficient hem synthesis, deficient erythropoiesis. Disorders according to 2) include, but are not limited to polycythemia vera, tumor-associated erythrocytosis, myelofibrosis, thrombocythemia. Disorders according to 3) include, but are not limited to vasculitis, thrombocytopenia, heparin-induced thrombocytopenia, thrombotic thrombocytopenic purpura, hemolytic-uremic syndrome, hereditary and acquired disorders of platelet function, hereditary coagulation disorders. Disorders according to 4) include, but are not limited to neutropenia, lymphocytopenia. Disorders according to 5) include, but are not limited to hypereosinophilia, idiopathic hypereosinophilic syndrome. Disorders according to 6) include, but are not limited to acute myeloic leukemia, acute lymphoblastic leukemia, chronic myelocytic leukemia, chronic lymphocytic leukemia, myelodysplastic syndrome. Disorders according to 7) include, but are not limited to Hodgkin's disease, non-Hodgkin's lymphoma, Burkitt's lymphoma, mycosis fungoides cutaneous T-cell lymphoma. Disorders according to 8) include, but are not limited to multiple myeloma, macroglobulinemia, heavy chain diseases. In extension of the preceding idiopathic thrombocytopenic purpura, iron deficiency anemia, megaloblastic anemia (vitamin B12 deficiency), aplastic anemia, thalassemia, malignant lymphoma bone marrow invasion, malignant lymphoma skin invasion, hemolytic uremic syndrome, giant platelet disease are considered to be hematological diseases too.

The human SGK is highly expressed in the following tissues of the hematological system: leukocytes (peripheral blood), bone marrow stromal cells, bone marrow CD15+ cells, neutrophils cord blood, neutrophils peripheral blood, spleen, spleen liver cirrhosis. The expression in the above mentioned tissues and in particular the differential expression between diseased tissue spleen liver cirrhosis and healthy tissue spleen demonstrates that the human SGK or mRNA can be utilized to diagnose of hematological diseases. Additionally the activity of the human SGK can be modulated to treat hematological disorders.

### Gastrointestinal and Liver Diseases

Gastrointestinal diseases comprise primary or secondary, acute or chronic diseases of the organs of the gastrointestinal tract which may be acquired or inherited, benign or malignant or metaplastic, and which may affect the organs of the gastrointestinal tract or the body as a whole. They comprise but are not limited to 1) disorders of the esophagus like achalasia, vigoruos achalasia, dysphagia, cricopharyngeal incoordination, pre-esophageal dysphagia, diffuse esophageal spasm, globus sensation, Barrett's metaplasia, gastroesophageal reflux, 2) disorders of the stomach and duodenum like functional dyspepsia, inflammation of the gastric mucosa, gastritis, stress gastritis, chronic erosive gastritis, atrophy of gastric glands, metaplasia of gastric tissues, gastric ulcers, duodenal ulcers, neoplasms of the stomach, 3) disorders of the pancreas like acute or chronic pancreatitis, insufficiency of the exocrinic or endocrinic tissues of the pancreas like steatorrhea, diabetes, neoplasms of the exocrine or endocrine pancreas like 3.1) multiple endocrine neoplasia syndrome, ductal adenocarcinoma, cystadenocarcinoma, islet cell tumors, insulinoma, gastrinoma, carcinoid tumors, glucagonoma, Zollinger-Ellison syndrome, Vipoma syndrome, malabsorption syndrome, 4) disorders of the bowel like chronic inflammatory diseases of the bowel, Crohn's disease, ileus, diarrhea and constipation, colonic inertia, megacolon, malabsorption syndrome, ulcerative colitis, 4.1) functional bowel disorders like irritable bowel syndrome, 4.2) neoplasms of the bowel like familial polyposis, adenocarcinoma, primary malignant lymphoma, carcinoid tumors, Kaposi's sarcoma, polyps, cancer of the colon and rectum.

Liver diseases comprise primary or secondary, acute or chronic diseases or injury of the liver which may be acquired or inherited, benign or malignant, and which may affect the liver or the body as a whole. They comprise but are not limited to disorders of the bilirubin metabolism, jaundice, syndroms of Gilbert's, Crigler-Najjar, Dubin-Johnson and Rotor; intrahepatic cholestasis, hepatomegaly, portal hypertension, ascites, Budd-Chiari syndrome, portal-systemic encephalopathy, fatty liver, steatosis, Reye's syndrome, liver diseases due to alcohol, alcoholic hepatitis or cirrhosis, fibrosis and cirrhosis, fibrosis and cirrhosis of the liver due to inborn errors of metabolism or exogenous substances, storage diseases, syndromes of Gaucher's, Zellweger's, Wilson's—disease, acute or chronic hepatitis, viral hepatitis and its variants, inflammatory conditions of the liver due to viruses, bacteria, fungi, protozoa, helminths; drug induced disorders of the liver, chronic liver diseases like primary sclerosing cholangitis, alpha1-antitrypsin-deficiency, primary biliary cirrhosis, postoperative liver disorders like postoperative intrahepatic cholestasis, hepatic granulomas, vascular liver disorders associated with systemic disease, benign or malignant neoplasms of the liver, disturbance of liver metabolism in the new-born or prematurely born.

The human SGK is highly expressed in the following tissues of the gastroenterological system: esophagus tumor, colon, colon tumor, ileum, ileum tumor, rectum, salivary gland, liver, liver tumor, HEP G2 cells. The expression in the above mentioned tissues demonstrates that the human SGK or mRNA can be utilized to diagnose of gastroenterological disorders. Additionally the activity of the human SGK can be modulated to treat gastroenterological disorders.

### Endocrine System and Hormones

The endocrine system consists of a group of organs whose main function is to produce and secrete hormones directly into the bloodstream. The major organs of the endocrine system are the hypothalamus, the pituitary gland, thyroid gland, the parathyroid glands, the islets of the pancreas, the adrenal glands, the testes, and the ovaries.

The hypothalamus secretes several hormones that stimulate the pituitary: Some trigger the release of pituitary hormones; others suppress the release of pituitary hormones.

The pituitary gland coordinates many functions of the other endocrine glands, but some pituitary hormones have direct effects.

The insulin-secreting cells of the pancreas respond to glucose and fatty acids. Parathyroid cells respond to calcium and phosphate. The adrenal medulla (part of the adrenal gland) responds to direct stimulation by the parasympathetic nervous system.

When endocrine glands malfunction, hormone levels in the blood can become abnormally high or low, disrupting body functions. Many disorders are caused by malfunction of the endocrine system or hormones. Examples of such disorders are presented in the following.

Diabetes mellitus is a disorder in which blood levels of glucose are abnormally high because the body doesn't release or use insulin adequately.

People with type I diabetes mellitus (insulin-dependent diabetes) produce little or no insulin at all. In type I diabetes more than 90 percent of the insulin-producing cells (beta cells) of the pancreas are permanently destroyed. The resulting insulin deficiency is severe, and to survive, a person with type I diabetes must regularly inject insulin.

In type II diabetes mellitus (non-insulin-dependent diabetes) the body develops resistance to insulin effects, resulting in a relative insulin deficiency.

The pancreas has two major functions: to secrete fluid containing digestive enzymes into the duodenum and to secrete the hormones insulin and glucagon. Chronic pancreatitis is a long-standing inflammation of the pancreas. Eventually, the insulin-secreting cells of the pancreas may be destroyed, gradually leading to diabetes. An insulinoma is a rare type of pancreatic tumor that secretes insulin. The symptoms of an insulinoma result from low blood glucose levels. A gastrinoma is a pancreatic tumor that produces excessive levels of the hormone gastrin, which stimulates the stomach to secrete acid and enzymes, causing peptic ulcers. The excess gastrin secreted by the gastrinoma causes symptoms, called the Zollinger-Ellison syndrome. A glucagonoma is a tumor that produces the hormone glucagon, which raises the level of glucose in the blood and produces a distinctive rash.

Diabetes insipidus is a disorder in which insufficient levels of antidiuretic hormone cause excessive thirst (polydipsia) and excessive production of very dilute urine (polyuria). Diabetes insipidus results from the decreased production of antidiuretic hormone (vasopressin).

The body has two adrenal glands. The medulla of the adrenal glands secretes hormones such as adrenaline (epinephrine) that affect blood pressure, heart rate, sweating, and other activities also regulated by the sympathetic nervous system. The cortex secretes many different hormones, including corticosteroids (cortisone-like hormones), androgens (male hormones), and mineralocorticoids, which control blood pressure and the levels of salt and potassium in the body.

A disease characterized by underactive adrenal glands is Addison's disease (adrenocortical insufficiency).

Several disorders are characterized by overactive Adrenal Glands. The causes can be changes in the adrenal glands themselves or overstimulation by the pituitary gland. Examples of these diseases are listed in the following.

Overproduction of androgenic steroids (testosterone and similar hormones, leads to virilization), overproduction of corticosteroids (causes could be tumors of the pituitary or the adrenal gland, results in Cushing's syndrome), Nelson's syndrome (developed by people who have both adrenal glands removed, characterized by an enlargement of the pituitary gland), Overproduction of aldosterone (hyperaldosteronism), Conn's syndrome (hyperaldosterism caused by a tumor), pheochromocytoma (a tumor that originating from the adrenal gland's chromaffin cells, causing overproduction of catecholamines).

The thyroid is a small gland located under the Adam's apple. It secretes thyroid hormones, which control the metabolic rate. The thyroid gland traps iodine and processes it into thyroid hormones. The euthyroid sick syndrome is characterized by lack of conversion of the T4 form of thyroid hormone to the T3 form. Hyperthyroidism (overactive thyroid gland, production of too much hormone) may have several causes. Thyroiditis (an inflammation of the thyroid gland), typically leads to a phase of hyperthyroidism. The inflammation may damage the thyroid gland, so that in later stages the disease is characterized by transient or permanent underactivity (hypothyroidism). Toxic thyroid nodules (adenomas) often produce thyroid hormone in large quantities. Toxic multinodular goiter (Plummer's disease) is a disorder in which there are many nodules. Graves' disease (toxic diffuse goiter) is believed to be caused by an antibody that stimulates the thyroid to produce too much thyroid hormone. In toxic nodular goiter, one or more nodules in the thyroid produce too much thyroid hormone and aren't under the control of thyroid-stimulating hormone. Secondary hyperthyroidism may (rarely) be caused by a pituitary tumor that secretes too much thyroid-stimulating hormone, by resistance of the pituitary to thyroid hormone, which results in the pituitary gland secreting too much thyroid-stimulating hormone, or by a hydatidiform mole in women. Thyroid storm is a sudden extreme overactivity of the thyroid gland is a life-threatening emergency requiring prompt treatment.

Hypothyroidism is a condition in which the thyroid gland is underactive and produces too little thyroid hormone. Very severe hypothyroidism is called myxedema. In Hashimoto's thyroiditis (autoimmune thyroiditis) the thyroid gland is often enlarged, and hypothyroidism results because the gland's functioning areas are gradually destroyed. Rarer causes of hypothyroidism include some inherited disorders which are caused by abnormalities of the enzymes in thyroid cells. In other rare disorders, either the hypothalamus or the pituitary gland fails to secrete enough of the hormone needed to stimulate normal thyroid function.

Other examples of Thyroiditis are silent lymphocytic thyroiditis, Hashimoto's thyroiditis, or subacute granulomatous thyroiditis.

Thyroid cancer is any one of four main types of malignancy of the thyroid: papillary, follicular, anaplastic, or medullary.

The pituitary is a pea-sized gland that sits in a bony structure (sella turcica) at the base of the brain. The sella turcica protects the pituitary but allows very little room for expansion. If the pituitary enlarges, it tends to push upward, often pressing on the areas of the brain that carry signals from the eyes, possibly resulting in headaches or impaired vision. The pituitary gland has two distinct parts: the anterior (front) and the posterior (back) lobes. The anterior lobe produces (secretes) hormones that ultimately control the function of the thyroid gland, adrenal glands, and reproductive organs (ovaries and testes); milk production (lactation) in the breasts; and overall body growth. It also produces hormones that cause the skin to darken and that inhibit pain sensations. The posterior lobe produces hormones that regulate water balance, stimulate the let-down of milk from the breasts in lactating women, and stimulate contractions of the uterus.

Examples for disorders of the pituitary gland are Empty Sella Syndrome; hypopituitarism (an underactive pituitary gland); acromegaly, which is excessive growth caused by over secretion of growth hormone, which is almost always caused by a benign pituitary tumor (adenoma); galactorrhea, which is the production of breast milk in men or in women who aren't breastfeeding, in both sexes, the most common cause of galactorrhea is a prolactin-producing tumor (prolactinoma) in the pituitary gland.

The human SGK is highly expressed in the following tissues of the endocrinological system: adrenal gland, thyroid, pancreas, pancreas liver cirrhosis. The expression in the above mentioned tissues and in particular the differential expression between diseased tissue pancreas liver cirrhosis and healthy tissue pancreas demonstrates that the human SGK or mRNA can be utilized to diagnose of endocrinological disorders. Additionally the activity of the human SGK can be modulated to treat endocrinological disorders.

### Cancer Disorders

Cancer disorders within the scope of this definition comprise any disease of an organ or tissue in mammals characterized by poorly controlled or uncontrolled multiplication of normal or abnormal cells in that tissue and its effect on the body as a whole. Cancer diseases within the scope of the definition comprise benign neoplasms, dysplasias, hyperplasias as well as neoplasms showing metastatic growth or any other transformations like e.g. leukoplakias which often precede a breakout of cancer. Cells and tissues are cancerous when they grow more rapidly than normal cells, displacing or spreading into the surrounding healthy tissue or any other tissues of the body described as metastatic growth, assume abnormal shapes and sizes, show changes in their nucleocytoplasmatic ratio, nuclear polychromasia, and finally may cease. Cancerous cells and tissues may affect the body as a whole when causing paraneoplastic syndromes or if cancer occurs within a vital organ or tissue, normal function will be impaired or halted, with possible fatal results. The ultimate involvement of a vital organ by cancer, either primary or metastatic, may lead to the death of the mammal affected. Cancer tends to spread, and the extent of its spread is usually related to an individual's chances of surviving the disease. Cancers are generally said to be in one of three stages of growth: early, or localized, when a tumor is still confined to the tissue of origin, or primary site; direct extension, where cancer cells from the tumour have invaded adjacent tissue or have spread only to regional lymph nodes; or metastasis, in which cancer cells have migrated to distant parts of the body from the primary site, via the blood or lymph systems, and have established secondary sites of infection. Cancer is said to be malignant because of its tendency to cause death if not treated. Benign tumors usually do not cause death, although they may if they interfere with a normal body function by virtue of their location, size, or paraneoplastic side effects. Hence benign tumors fall under the definition of cancer within the scope of this definition as well. In general, cancer cells divide at a higher rate than do normal cells, but the distinction between the growth of cancerous and normal tissues is not so much the rapidity of cell division in the former as it is the partial or complete loss of growth restraint in cancer cells and their failure to differentiate into a useful, limited tissue of the type that characterizes the functional equilibrium of growth of normal tissue. Cancer tissues may express certain molecular receptors and probably are influenced by the host's susceptibility and immunity and it is known that certain cancers of the breast and prostate, for example, are considered dependent on specific hormones for their existence. The term “cancer” under the scope of the definition is not limited to simple benign neoplasia but comprises any other benign and malign neoplasia like 1) Carcinoma, 2) Sarcoma, 3) Carcinosarcoma, 4) Cancers of the blood-forming tissues, 5) tumors of nerve tissues including the brain, 6) cancer of skin cells. Cancer according to 1) occurs in epithelial tissues, which cover the outer body (the skin) and line mucous membranes and the inner cavitary structures of organs e.g. such as the breast, lung, the respiratory and gastrointestinal tracts, the endocrine glands, and the genitourinary system. Ductal or glandular elements may persist in epithelial tumors, as in adenocarcinomas like e.g. thyroid adenocarcinoma, gastric adenocarcinoma, uterine adenocarcinoma. Cancers of the pavement-cell epithelium of the skin and of certain mucous membranes, such as e.g. cancers of the tongue, lip, larynx, urinary bladder, uterine cervix, or penis, may be termed epidermoid or squamous-cell carcinomas of the respective tissues and are in the scope of the definition of cancer as well. Cancer according to 2) develops in connective tissues, including fibrous tissues, adipose (fat) tissues, muscle, blood vessels, bone, and cartilage like e.g. osteogenic sarcoma; liposarcoma, fibrosarcoma, synovial sarcoma. Cancer according to 3) is cancer that develops in both epithelial and connective tissue. Cancer disease within the scope of this definition may be primary or secondary, whereby primary indicates that the cancer originated in the tissue where it is found rather than was established as a secondary site through metastasis from another lesion. Cancers and tumor diseases within the scope of this definition may be benign or malign and may affect all anatomical structures of the body of a mammal. By example but not limited to they comprise cancers and tumor diseases of I) the bone marrow and bone marrow derived cells (leukemias), II) the endocrine and exocrine glands like e.g. thyroid, parathyroid, pituitary, adrenal glands, salivary glands, pancreas Ill) the breast, like e.g. benign or malignant tumors in the mammary glands of either a male or a female, the mammary ducts, adenocarcinoma, medullary carcinoma, comedo carcinoma, Paget's disease of the nipple, inflammatory carcinoma of the young woman, IV) the lung, V) the stomach, VI) the liver and spleen, VII) the small intestine, VIII) the colon, IX) the bone and its supportive and connective tissues like malignant or benign bone tumour, e.g. malignant osteogenic sarcoma, benign osteoma, cartilage tumors; like malignant chondrosarcoma or benign chondroma; bone marrow tumors like malignant myeloma or benign eosinophilic granuloma, as well as metastatic tumors from bone tissues at other locations of the body; X) the mouth, throat, larynx, and the esophagus, XI) the urinary bladder and the internal and external organs and structures of the urogenital system of male and female like ovaries, uterus, cervix of the uterus, testes, and prostate gland, XII) the prostate, XIII) the pancreas, like ductal carcinoma of the pancreas; XIV) the lymphatic tissue like lymphomas and other tumors of lymphoid origin, XV) the skin, XVI) cancers and tumor diseases of all anatomical structures belonging to the respiration and respiratory systems including thoracal muscles and linings, XVII) primary or secondary cancer of the lymph nodes XVII) the tongue and of the bony structures of the hard palate or sinuses, XVIV) the mouth, cheeks, neck and salivary glands, XX) the blood vessels including the heart and their linings, XXI) the smooth or skeletal muscles and their ligaments and linings, XXII) the peripheral, the autonomous, the central nervous system including the cerebellum, XXIII) the adipose tissue.

The human SGK is highly expressed in the following cancer cells and tissues: HUVEC cells, HeLa cells, esophagus tumor, colon tumor, ileum tumor, liver tumor, DEP G2 cells, uterus tumor, ovary tumor, breast tumor, kidney tumor. The expression in the above mentioned tissues and in particular the differential expression between diseased tissue esophagus tumor and healthy tissue esophagus, between diseased tissue colon tumor and healthy tissue colon, between diseased tissue ileum tumor and healthy tissue ileum, between diseased tissue liver tumor and healthy tissue liver, between diseased tissue HEP G2 cells and healthy tissue liver, between diseased tissue uterus tumor and healthy tissue uterus, between diseased tissue ovary tumor and healthy tissue ovary, between diseased tissue breast tumor and healthy tissue breast, between diseased tissue kidney tumor and healthy tissue kidney demonstrates that the human SGK or mRNA can be utilized to diagnose of cancer. Additionally, the activity of the human SGK can be modulated to treat cancer.

Therefore, disclosed herein is a method for treating a cancer in a subject, comprising administering to the subject an agent that blocks the binding of endogenous SGK1 to endogenous GILZ. For example, the cancer of the disclosed methods can be any cell in a subject undergoing unregulated growth, invasion, or metastasis. In some embodiments, the cancer can be any neoplasm or tumor for which radiotherapy is currently used. Alternatively, the cancer can be a neoplasm or tumor that is not sufficiently sensitive to radiotherapy using standard methods. Thus, the cancer can be a sarcoma, lymphoma, leukemia, carcinoma, blastoma, or germ cell tumor. A representative but non-limiting list of cancers that the disclosed compositions can be used to treat include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

### Inflammatory Diseases

Inflammatory diseases comprise diseases triggered by cellular or non-cellular mediators of the immune system or tissues causing the inflammation of body tissues and subsequently producing an acute or chronic inflammatory condition. Examples for such inflammatory diseases are hypersensitivity reactions of type I-IV, for example but not limited to hypersensitivity diseases of the lung including asthma, atopic diseases, allergic rhinitis or conjunctivitis, angioedema of the lids, hereditary angioedema, antireceptor hypersensitivity reactions and autoimmune diseases, Hashimoto's thyroiditis, systemic lupus erythematosus, Goodpasture's syndrome, pemphigus, myasthenia gravis, Grave's and Raynaud's disease, type B insulin-resistant diabetes, rheumatoid arthritis, psoriasis, Crohn's disease, scleroderma, mixed connective tissue disease, polymyositis, sarcoidosis, glomerulonephritis, acute or chronic host versus graft reactions.

The human SGK is highly expressed in the following tissues of the immune system and tissues responsive to components of the immune system as well as in the following tissues responsive to mediators of inflammation: pancreas liver cirrhosis, leukocytes (peripheral blood), bone marrow CD15+ cells, neutrophils cord blood, neutrophils peripheral blood, spleen liver cirrhosis. The expression in the above mentioned tissues and in particular the differential expression between diseased tissue pancreas liver cirrhosis and healthy tissue pancreas, between diseased tissue spleen liver cirrhosis and healthy tissue spleen demonstrates that the human SGK or mRNA can be utilized to diagnose of inflammatory diseases. Additionally the activity of the human SGK can be modulated to treat inflammatory diseases.

### Disorders Related to Pulmonology

Asthma is thought to arise as a result of interactions between multiple genetic and environmental factors and is characterized by three major features: 1) intermittent and reversible airway obstruction caused by bronchoconstriction, increased mucus production, and thickening of the walls of the airways that leads to a narrowing of the airways, 2) airway hyperresponsiveness, and 3) airway inflammation. Certain cells are critical to the inflammatory reaction of asthma and they include T cells and antigen presenting cells, B cells that produce IgE, and mast cells, basophils, eosinophils, and other cells that bind IgE. These effector cells accumulate at the site of allergic reaction in the airways and release toxic products that contribute to the acute pathology and eventually to tissue destruction related to the disorder. Other resident cells, such as smooth muscle cells, lung epithelial cells, mucus-producing cells, and nerve cells may also be abnormal in individuals with asthma and may contribute to its pathology. While the airway obstruction of asthma, presenting clinically as an intermittent wheeze and shortness of breath, is generally the most pressing symptom of the disease requiring immediate treatment, the inflammation and tissue destruction associated with the disease can lead to irreversible changes that eventually make asthma a chronic and disabling disorder requiring long-term management.

Chronic obstructive pulmonary (or airways) disease (COPD) is a condition defined physiologically as airflow obstruction that generally results from a mixture of emphysema and peripheral airway obstruction due to chronic bronchitis [Botstein, 1980]. Emphysema is characterised by destruction of alveolar walls leading to abnormal enlargement of the air spaces of the lung. Chronic bronchitis is defined clinically as the presence of chronic productive cough for three months in each of two successive years. In COPD, airflow obstruction is usually progressive and is only partially reversible. By far the most important risk factor for development of COPD is cigarette smoking, although the disease does also occur in non-smokers.

The human SGK is highly expressed in the following tissues of the respiratory system: leukocytes (peripheral blood), bone marrow CD15+ cells, neutrophils cord blood, neutrophils peripheral blood, fetal lung, fetal lung fibroblast IMR-90 cells. The expression in the above mentioned tissues and in particular the differential expression between diseased tissue fetal lung fibroblast IMR-90 cells and healthy tissue fetal lung demonstrates that the human SGK or mRNA can be utilized to diagnose of respiratory diseases. Additionally the activity of the human SGK can be modulated to treat those diseases.

### Disorders Related to Urology

Genitourinary disorders comprise benign and malign disorders of the organs constituting the genitourinary system of female and male, renal diseases like acute or chronic renal failure, immunologically mediated renal diseases like renal transplant rejection, lupus nephritis, immune complex renal diseases, glomerulopathies, nephritis, toxic nephropathy, obstructive uropathies like benign prostatic hyperplasia (BPH), neurogenic bladder syndrome, urinary incontinence like urge-, stress-, or overflow incontinence, pelvic pain, and erectile dysfunction.

The human SGK is highly expressed in the following urological tissues: spinal cord, prostate, prostate BPH, bladder, fetal kidney, kidney, kidney tumor. The expression in the above mentioned tissues and in particular the differential expression between diseased tissue prostate BPH and healthy tissue prostate demonstrates that the human SGK or mRNA can be utilized to diagnose of urological disorders. Additionally the activity of the human SGK can be modulated to treat urological disorders.

### Metabolic Disorders

Metabolic diseases are defined as conditions which result from an abnormality in any of the chemical or biochemical transformations and their regulating systems essential to producing energy, to regenerating cellular constituents, to eliminating unneeded products arising from these processes, and to regulate and maintain homeostasis in a mammal regardless of whether acquired or the result of a genetic transformation. Depending on which metabolic pathway is involved, a single defective transformation or disturbance of its regulation may produce consequences that are narrow, involving a single body function, or broad, affecting many organs, organ-systems or the body as a whole. Diseases resulting from abnormalities related to the fine and coarse mechanisms that affect each individual transformation, its rate and direction or the availability of substrates like amino acids, fatty acids, carbohydrates, minerals, cofactors, hormones, regardless whether they are inborn or acquired, are well within the scope of the definition of a metabolic disease according to this application.

Metabolic diseases often are caused by single defects in particular biochemical pathways, defects that are due to the deficient activity of individual enzymes or molecular receptors leading to the regulation of such enzymes. Hence in a broader sense disturbances of the underlying genes, their products and their regulation lie well within the scope of this definition of a metabolic disease. For example, but not limited to, metabolic diseases may affect 1) biochemical processes and tissues ubiquitous all over the body, 2) the bone, 3) the nervous system, 4) the endocrine system, 5) the muscle including the heart, 6) the skin and nervous tissue, 7) the urogenital system, 8) the homeostasis of body systems like water and electrolytes. For example, but not limited to, metabolic diseases according to 1) comprise obesity, amyloidosis, disturbances of the amino acid metabolism like branched chain disease, hyperaminoacidemia, hyperaminoaciduria, disturbances of the metabolism of urea, hyperammonemia, mucopolysaccharidoses e.g. Maroteaux-Lamy syndrom, storage diseases like glycogen storage diseases and lipid storage diseases, glycogenosis diseases like Cori's disease, malabsorption diseases like intestinal carbohydrate malabsorption, oligosaccharidase deficiency like maltase-, lactase-, sucrase-insufficiency, disorders of the metabolism of fructose, disorders of the metabolism of galactose, galactosaemia, disturbances of carbohydrate utilization like diabetes, hypoglycemia, disturbances of pyruvate metabolism, hypolipidemia, hypolipoproteinemia, hyperlipidemia, hyperlipoproteinemia, carnitine or carnitine acyltransferase deficiency, disturbances of the porphyrin metabolism, porphyrias, disturbances of the purine metabolism, lysosomal diseases, metabolic diseases of nerves and nervous systems like gangliosidoses, sphingolipidoses, sulfatidoses, leucodystrophies, Lesch-Nyhan syndrome. For example, but not limited to, metabolic diseases according to 2) comprise osteoporosis, osteomalacia like osteoporosis, osteopenia, osteogenesis imperfecta, osteopetrosis, osteonecrosis, Paget's disease of bone, hypophospliatemia. For example, but not limited to, metabolic diseases according to 3) comprise cerebellar dysfunction, disturbances of brain metabolism like dementia, Alzheimer's disease, Huntington's chorea, Parkinson's disease, Pick's disease, toxic encephalopathy, demyelinating neuropathies like inflammatory neuropathy, Guillain-Barré syndrome. For example, but not limited to, metabolic diseases according to 4) comprise primary and secondary metabolic disorders associated with hormonal defects like any disorder stemming from either an hyperfunction or hypofunction of some hormone-secreting endocrine gland and any combination thereof. They comprise Sipple's syndrome, pituitary gland dysfunction and its effects on other endocrine glands, such as the thyroid, adrenals, ovaries, and testes, acromegaly, hyper- and hypothyroidism, euthyroid goiter, euthyroid sick syndrome, thyroiditis, and thyroid cancer, over- or underproduction of the adrenal steroid hormones, adrenogenital syndrome, Cushing's syndrome, Addison's disease of the adrenal cortex, Addison's pernicious anemia, primary and secondary aldosteronism, diabetes insipidus, carcinoid syndrome, disturbances caused by the dysfunction of the parathyroid glands, pancreatic islet cell dysfunction, diabetes, disturbances of the endocrine system of the female like estrogen deficiency, resistant ovary syndrome. For example, but not limited to, metabolic diseases according to 5) comprise muscle weakness, myotonia, Duchenne's and other muscular dystrophies, dystrophia myotonica of Steinert, mitochondrial myopathies like disturbances of the catabolic metabolism in the muscle, carbohydrate and lipid storage myopathies, glycogenoses, myoglobinuria, malignant hyperthermia, polymyalgia rheumatica, dermatomyositis, primary myocardial disease, cardiomyopathy. For example, but not limited to, metabolic diseases according to 6) comprise disorders of the ectoderm, neurofibromatosis, scleroderma and polyarteritis, Louis-Bar syndrome, von Hippel-Lindau disease, Sturge-Weber syndrome, tuberous sclerosis, amyloidosis, porphyria. For example, but not limited to, metabolic diseases according to 7) comprise sexual dysfunction of the male and female. For example, but not limited to, metabolic diseases according to 8) comprise confused states and seizures due to inappropriate secretion of antidiuretic hormone from the pituitary gland, Liddle's syndrome, Bartter's syndrome, Fanconi's syndrome, renal electrolyte wasting, diabetes insipidus.

The human SGK is highly expressed in the following metabolic disease related tissues: thyroid, pancreas, pancreas liver cirrhosis, liver, HEP G2 cells, spleen liver cirrhosis. The expression in the above mentioned tissues and in particular the differential expression between diseased tissue pancreas liver cirrhosis and healthy tissue pancreas, between diseased tissue spleen liver cirrhosis and healthy tissue spleen demonstrates that the human SGK or mRNA can be utilized to diagnose of metabolic diseases. Additionally the activity of the human SGK can be modulated to treat metabolic diseases.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

### Background

ER Stress and Canonical ER associated Protein Degradation (ERAD): ER stress occurs in many forms of heart disease, including pathologies related to pressure overload-induced cardiac hypertrophy and heart failure (Blackwood E A, et al. Cells. 2020 9; Glembotski C C, et al. J Am Coll Cardiol. 2019 73:1807-1810). In all cells, including cardiac myocytes, pathology can alter the ER in ways that impairs ER protein-folding, causing ER stress and subsequent activation of the ER stress response (Glembotski C C. J Mol Cell Cardiol. 2008 44:453-9; Ron D and Walter P. Nat Rev Mol Cell Biol. 2007 8:519-29). Initially, the ER stress response restores proper ER protein-folding by inducing genes encoding ER proteins responsible for protein-folding. Although the details vary by cell type and the nature of the ER stress, initial ER stress is generally adaptive, favoring survival, while chronic ER stress is maladaptive, favoring cell death (Karagoz G E, et al. Cold Spring Harb Perspect Biol. 2019). Depending on the timing and nature of the stress, this response can be adaptive or maladaptive.

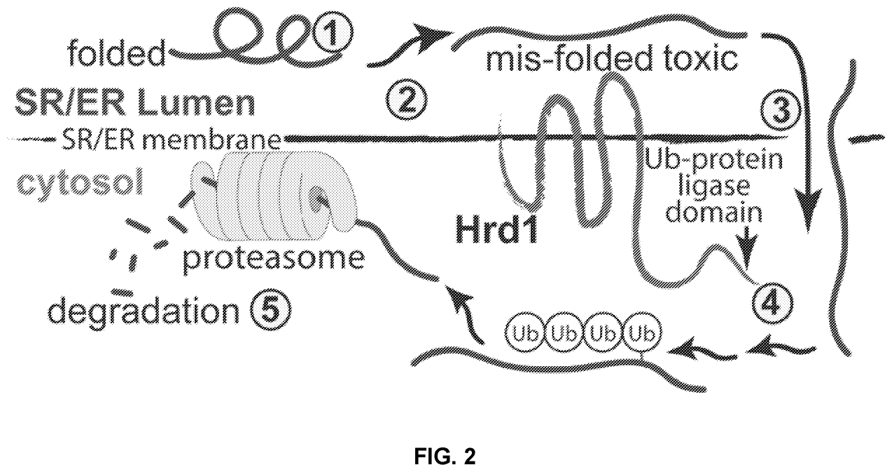

The canonical role for ER associated degradation (ERAD) is to degrade proteins that misfold in the ER. However, since proteins cannot be degraded in the ER, misfolded proteins are translocated out of the ER, then ubiquitylated and degraded by proteasomes outside the ER (Brodsky J L. Cell. 2012 151:1163-7) (FIG. 2). Ubiquitylation of misfolded ER proteins involves transmembrane ER E3 ubiquitin ligases; while there are hundreds of E3 ubiquitin ligases, only a few are ER transmembrane proteins (Brodsky J L. Cell. 2012 151:1163-7). In the mouse heart (Doroudgar S, et al. Circulation Research. 2015 117:536-546), one such ER E3 ubiquitin ligase, Hrd1, increases ERAD, and diminishes pressure overload-induced cardiac hypertrophy; however, the connection between ERAD and cardiac myocyte growth was not investigated.

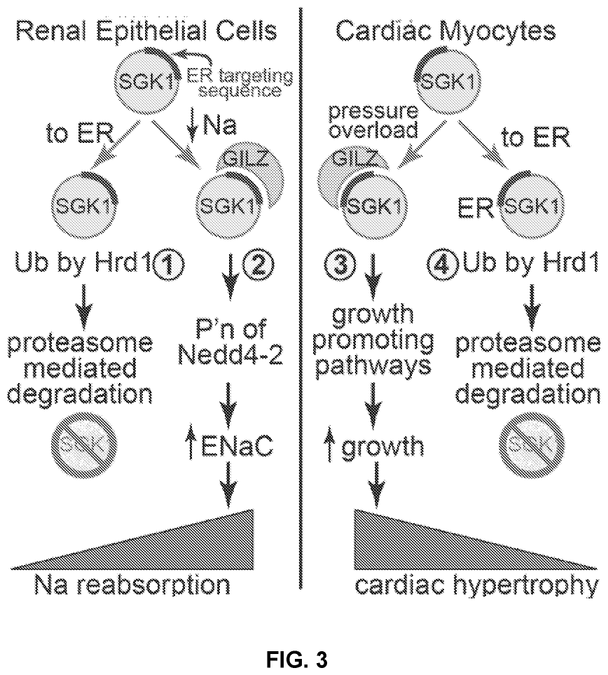

Scientific Premises Supporting Roles for Non-canonical ERAD, SGK1 and GILZ: This proposal concerns whether SGK1 mechanistically links ERAD and heart growth. SGK1, reviewed in Maestro I, et al. Expert Opin Ther Targets. 2020 24:231-243, is an ˜49 kD serum and glucocorticoid-inducible member of the AGC family of protein kinases (Webster M K, et al. J Biol Chem. 1993 268:11482-5; Webster M K, et al. Mol Cell Biol. 1993 13:2031-40), being most similar (54% homologous) to AKT (Lang F and Cohen P. Sci STKE. 2001 2001:rel7). Although AKT and SGK1 exhibit some substrate overlap, they serve distinct functions (Murray J T, et al. FEBS Lett. 2005 579:991-4). SGK1 has been well-studied in epithelial cells (Loffing J, et al. Annu Rev Physiol. 2006 68:461-90), and in cancer cells (Bruhn M A, et al. Growth Factors. 2010 28:394-408), but less studied in cardiac myocytes (Aoyama T, et al. Circulation. 2005 111:1652-9; Lister K, et al. Cardiovasc Res. 2006 70:555-65). In renal epithelial cells, SGK1 increases Na reabsorption. In fact, unlike AKT, SGK1 regulates the levels and activities of several solute transporters (Lang F, et al. Curr Opin Nephrol Hypertens. 2009 18:439-48). In cancer cells, SGK1 increases proliferation (Basnet R, et al. Acta Pharm Sin B. 2018 8:767-771). SGK1 has been studied in cultured cardiac myocytes and in the heart, mostly using SGK1 overexpression and/or small molecule SGK1 inhibitors, showing that SGK1 increases cultured myocyte growth and Na channel activity in vivo (Aoyama T, et al. Circulation. 2005 111:1652-9; Das S, et al. Circulation. 2012 126:2208-19; Lister K, et al. Cardiovasc Res. 2006 70:555-65; Boehmer C, et al. Cardiovasc Res. 2003 57:1079-84). However, little is known about what regulates how and when SGK1 contributes to pathological cardiac hypertrophy. In terms of signaling, in cancer cells SGK1 phosphorylates NRDG1 (Murray J T, et al. Biochem J. 2004 384:477-88), Nedd4-2 (Debonneville C, et al. EMBO J. 2001 20:7052-9), Fox03a (Tran H, et al. Sci STKE. 2003 2003:RE5) and GSK3P (Aoyama T, et al. Circulation. 2005 111:1652-9), and activates mTOR (Lister K, et al. Cardiovasc Res. 2006 70:555-65). SGK1 is regulated at the transcriptional level and post-transcriptional levels; however, it is likely that ERAD plays a major role in post-translational regulation of SGK1, which is therefore, the focus of this proposal. Like many kinases, SGK1 conditionally localizes to various regions of cells (Maestro I, et al. Expert Opin Ther Targets. 2020 24:231-243). But, in contrast to other kinases, at least in renal epithelial cells, SGK1 conditionally localizes to the cytosolic face of the ER, where it interacts with Hrd1 (Arteaga M F, et al. Proc Natl Acad Sci USA. 2006 103:11178-83; Bogusz A M, et al. FEBS J. 2006 273:2913-28); this intriguing interaction has not been studied in the heart. In renal cells, when plasma Na is sufficient, SGK1 localizes to the ER, where it is ubiquitylated by Hrd1, then degraded by proteasomes (Arteaga M F, et al. Proc Natl Acad Sci USA. 2006 103:11178-83) (FIG. 3, \textcircled{1}). A unique N-terminal motif in SGK1 (FIG. 3) targets it to the ER (Bogusz A M, et al. FEBS J. 2006 273:2913-28; Brickley D R, et al. J Biol Chem. 2002 277:43064-70). Low plasma sodium induces GILZ, a ˜14 kD glucocorticoid-inducible transcription factor (D'Adamio F, et al. Immunity. 1997 7:803-12), that has been shown to have a non-transcriptional role, where it binds to SGK1 and blocks SGK1 localization to the ER (Soundararajan R, et al. Proc Natl Acad Sci USA. 2009 106:7804-9). Thus, GILZ diverts SGK1 away from the ER, thereby increasing SGK1 phosphorylation of the E3 Ub'n ligase, Nedd4-2 (FIG. 3, \textcircled{2}), which increases levels of the epithelial Na channel, ENaC, thus increasing Na reabsorption (Pao A C, et al. Am J Physiol Renal Physiol. 2007 292:F1741-50; Arteaga M F, et al. Mol Biol Cell. 2007 18:2072-80).

SGK1 is disclosed herein to be a major inducer of pressure overload-induced cardiac pathology. During pressure overload, SGK1 levels, and thus, SGK1-mediated cardiac hypertrophy and subsequent pathology, are increased by GILZ-dependent diversion of SGK1 away from the ER, which decreases SGK1 degradation by non-canonical ERAD (FIG. 3, \textcircled{3}\textcircled{4}). Moreover, it is proposed that ectopic expression of an SGK1 peptide disrupts the GILZ-SGK1 interaction, increases SGK1 degradation, thus decreasing SGK1-mediated cardiac hypertrophy and subsequent pathology.

### Results

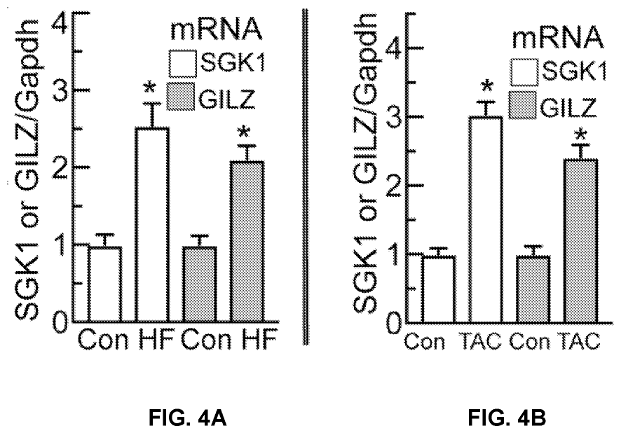

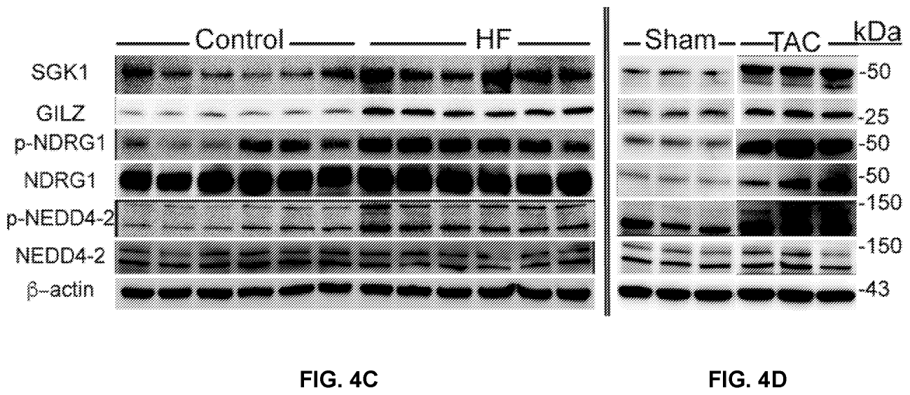

SGK1 and GILZ are induced in human and in mouse HF: As an initial step toward determining the significance of SGK1 and GILZ in cardiac pathology, the levels of SGK1 and GILZ mRNA were assessed in human hypertrophic cardiomyopathy heart failure (HF) samples, showing that both were significantly induced in the HF samples, compared to control (FIG. 4A). Immunoblots for endogenous SGK1 and GILZ in the same samples also showed increased expression of both, and probing for p-NDRG1 and p-NEDD4-2, direct targets of SGK1 (Murray J T, et al. Biochem J. 2004 384:477-88; Debonneville C, et al. EMBO J. 2001 20:7052-9), showed increased SGK1-dependent signaling in human HF samples (FIG. 4C). To examine whether the results in diseased human heart were recapitulated in a mouse model of hypertrophic cardiomyopathy, the effects of transaortic constriction (TAC)-induced pressure overload were examined, where SGK1 and GILZ mRNA (FIG. 4B), as well as SGK1 and GILZ protein and downstream signaling were increased 6W after TAC (FIG. 4D).

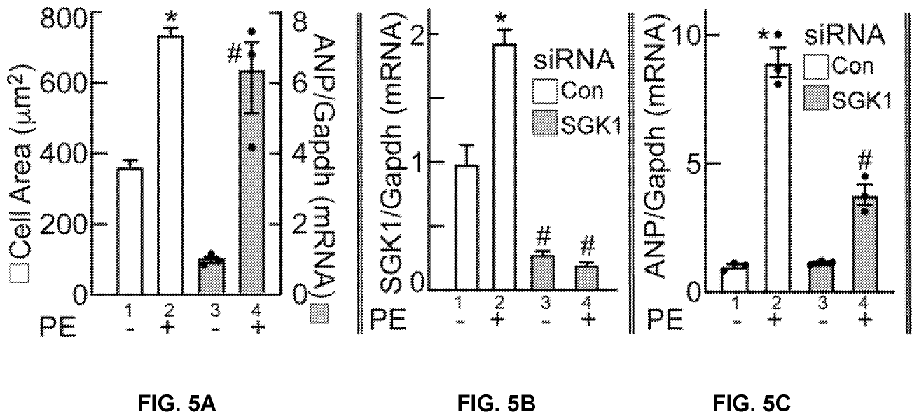

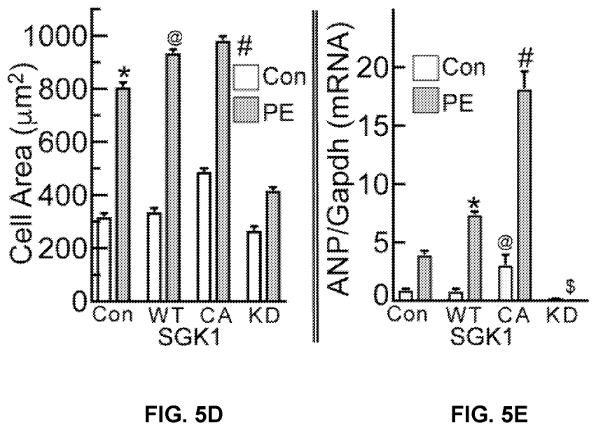

SGK1 is induced during, and required for NRVM growth: To begin to address mechanistically whether SGK1 is required for cardiac hypertrophy, its expression was examined in NRVM treated±the α₁-adrenergic receptor agonist, phenylephrine (PE), a well-known promoter of growth that mimics the pathological hypertrophy (Simpson P. Circ Res. 1985 56:884-94). As expected, PE increased NRVM growth (FIG. 5A bars 1,2), as well as mRNA for the fetal gene, ANP (FIG. 5A, bars 3,4), a molecular marker of cardiac hypertrophy (Garcia R, et al. Biochem Biophys Res Commun. 1987 145:532-41; Lee R T, et al. J Clin Invest. 1988 81:431-4). Note that since ANP induction is so strongly correlated with pathological cardiac hypertrophy, in some of the preliminary experiments in NRVM here, ANP was used as a reporter of cardiac myocyte growth. PE also increased SGK1 mRNA levels in NRVM (FIG. 5B, bars 1,2). Note that since there are no reliable antibodies for detecting SGK1 in rat cardiac myocytes, endogenous SGK1 was not able to be examined at the protein level in NRVM, so SGK1 mRNA levels were use as indicators of endogenous SGK1 expression in this model. To determine whether SGK1 is required for NRVM growth, SGK1 was knocked down using siRNA (FIG. 5B, bars 3,4). SGK1 knockdown significantly blunted ANP induction by PE (FIG. 5C, bar 2 vs 4). Also tested was whether ectopic expression of FLAG-SGK1 could enhance growth; accordingly, adenovirus (AdV) encoding wild type (WT) SGK1 was made, showing that it increased NRVM growth and ANP induction (FIG. 5D, 5E Con vs WT). NRVM growth was even more pronounced by an AdV encoding constitutively active SGK1, SGK1-CA (FIG. 5D, 5E Con vs CA); however, AdV encoding kinase dead SGK1, SGK1-KD, did not increase NRVM growth or ANP mRNA (FIG. 5D, 5E Con vs KD), and actually decreased ANP mRNA in both Con and PE, behaving like a dominant negative.

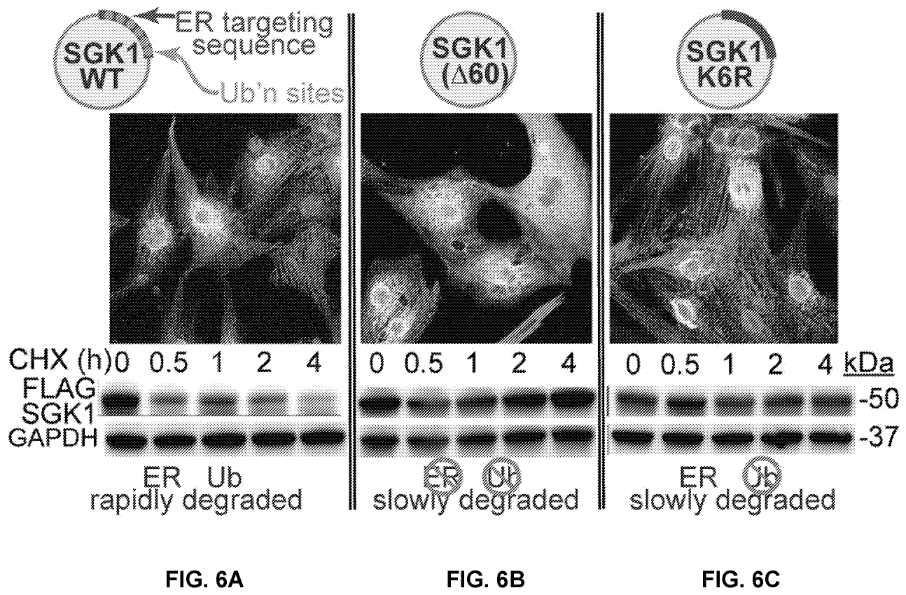

SGK1 is rapidly degraded by non-canonical ERAD in cardiac myocytes: To examine the hypothesis that SGK1 is rapidly degraded in cardiac myocytes, an AdV was made encoding FLAG-WT-SGK1, FLAG-SGK1-Δ(1-60) [deletion of N-terminal 60 amino acids, which include the ER targeting sequence and all 6 Ub'n sites], and FLAG-SGK1-K6R [all Ub'n sites mutated out](FIG. 6, top diagram). Immunocytofluorescence (ICF) showed that, as anticipated, WT, A60 and K6R are localized to the ER (FIG. 6A), cytosol (6B) and ER (6C), respectively. A cycloheximide (CHX) chase experiment assessed the degradation rates of the FLAG-SGK1 proteins. WT-SGK1 was degraded rapidly (FIG. 6A), while A60 (6B) and K6R (6C) were degraded very slowly. Thus, SGK1 degradation in cardiac myocytes is increased by its localization to the ER, and it requires the 6 lysine residues known to be ubiquitylated.

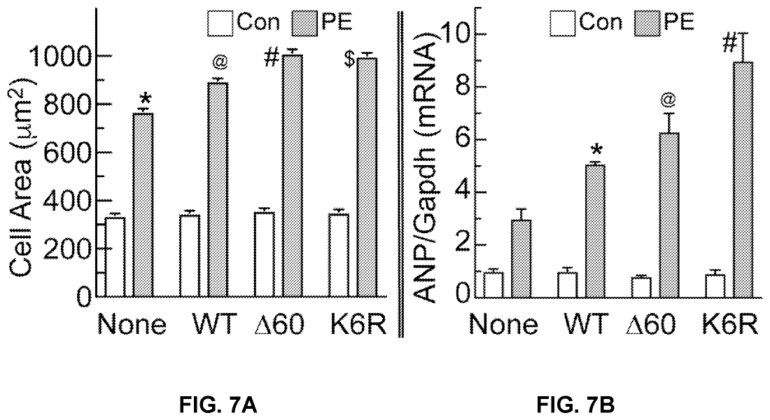

Relationship between SGK1 degradation rate and cardiac myocyte growth: To test the hypothesis that SGK1 degradation at the ER reduces its effects on cardiac myocyte growth, NRVM were infected with AdV-Con, WT, A60 or K6R, then treated±PE. Without PE, myocyte size and ANP mRNA were relatively unaffected by each form of SGK1; however, PE-mediated growth, and ANP mRNA, levels were increased by SGK1-WT, and even more so by A60 and K6R (FIG. 7A, 7B). Thus, SGK1 degradation at the ER by non-canonical ERAD decreases its effects on NRVM growth.

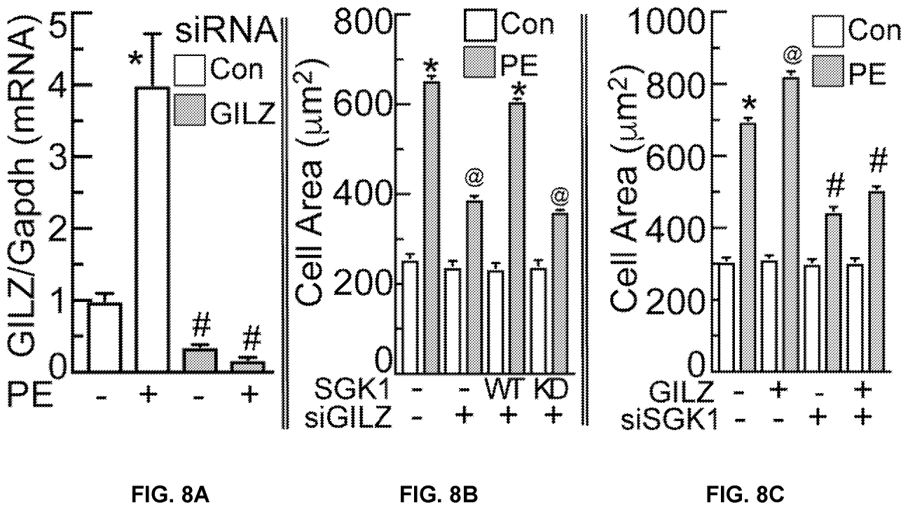

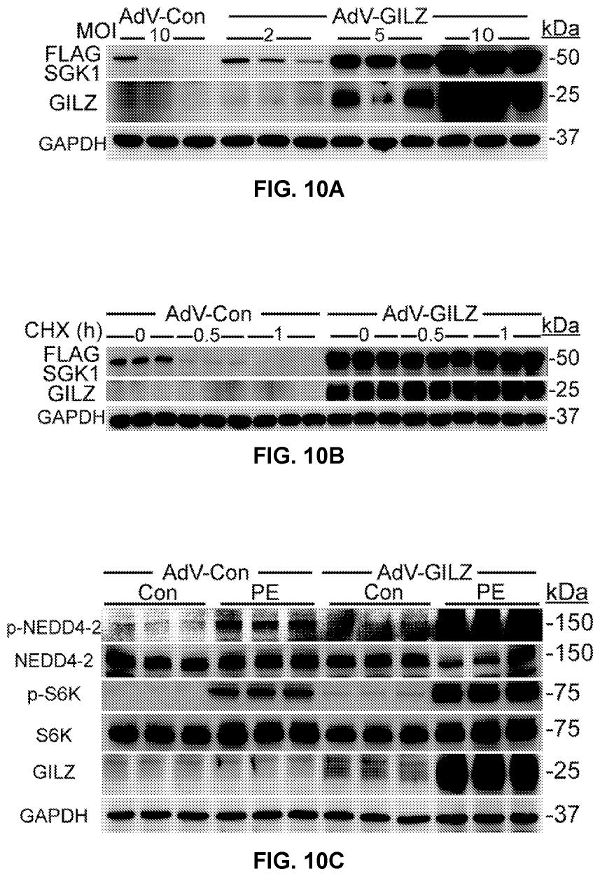

GILZ is induced during, and required for NRVM growth: Like SGK1, GILZ was induced in failing human and mouse hearts (FIG. 4). Accordingly, examined was whether PE could induce GILZ in NRVM, as it induces SGK1 (FIG. 5B). PE strongly upregulated GILZ mRNA in NRVM (FIG. 8A, left). To examine GILZ function upon induction in NRVM, GILZ knock down (FIG. 8A, right) significantly decreased PE-mediated NRVM growth, which could be overcome by co-transfection of SGK1-WT but not −KD (FIG. 8B). To test GILZ gain-of-function, we generated AdV encoding GILZ, which expressed the appropriate protein (see FIGS. 10A and 10B, below). AdV-GILZ significantly increased PE-mediated NRVM growth in an SGK1-dependent manner (FIG. 8C). Thus, GILZ and SGK1 are both induced by, and both are required for PE-mediated NRVM growth.

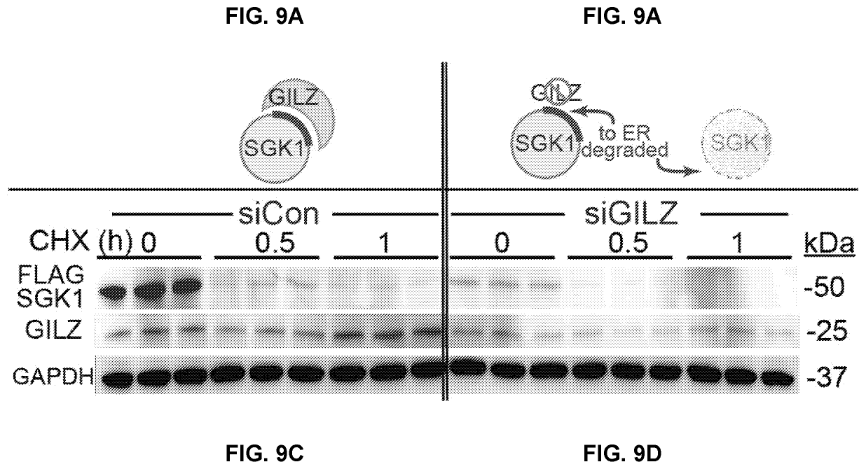

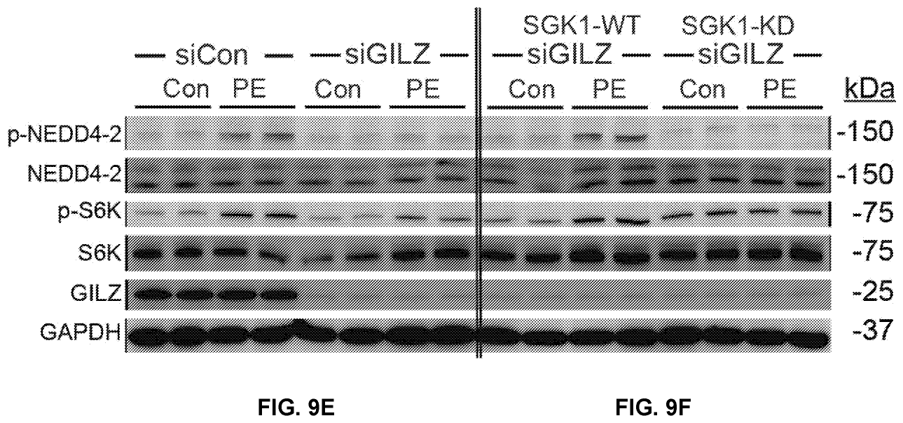

GILZ knockdown accelerates SGK1 degradation: Next determined was whether GILZ protects SGK1 from ERAD-mediated degradation by examining the effects of GILZ siRNA on SGK1 degradation (FIG. 9A, 9B). A CHX chase experiment showed that GILZ siRNA strongly accelerated SGK1 degradation (FIG. 9C, 9D). Next assessed was how GILZ siRNA affects SGK1 signaling to p-NEDD4-2 and p-S6K. GILZ siRNA decreased PE-mediated increases in both p-NEDD4-2 and p-S6K (FIG. 9E); this blockade was restored by co-infecting with AdV-FLAG-SGK1-WT, but not KD (FIG. 9F). The results in FIGS. 8 and 9 support the concept that GILZ is necessary for NRVM growth because it protects SGK1 from ERAD-mediated degradation.

Ectopic expression of GILZ slows SGK1 degradation: To further examine the relationship between GILZ, SGK1 and cardiac myocyte growth, GILZ was ectopically expressed in NRVM using AdV-GILZ and signaling downstream of SGK1 was examined by immunoblotting. When NRVM were treated with increasing amounts of AdV-GILZ and a single dose of AdV-FLAG-SGK1-WT, there was the anticipated AdV-dose-dependent increase in the amounts of GILZ expressed, as well as increased amounts of FLAG-SGK1-WT (FIG. 10A), the latter being consistent with decreased SGK1 degradation by GILZ. To demonstrate this further, a CHX chase experiment showed that GILZ slowed FLAG-SGK1 degradation (FIG. 10B). The GILZ-mediated decreases in FLAG-SGK1 degradation were also reflected as substantially increased SGK1 signaling to p-NEDD4-2 and p-S6K in PE-treated NRVM (FIG. 10C).

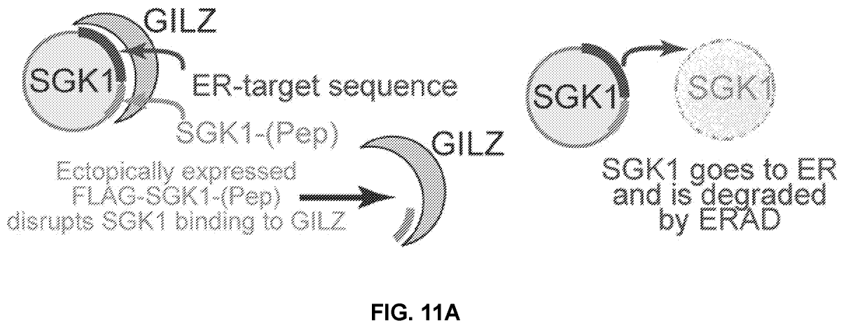

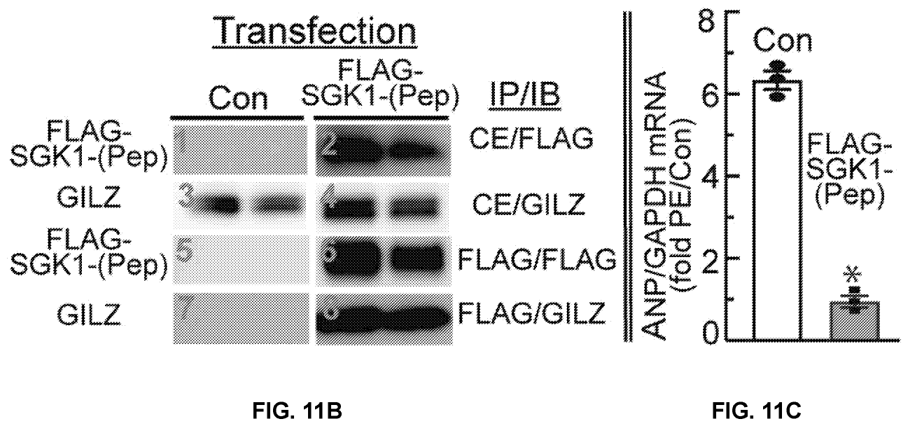

SGK1(122-157) interrupts SGK1/GILZ interaction and decreases cardiac myoctye growth: To show that GILZ interaction with SGK1 is required for SGK1 degradation, a search was conducted for the region of SGK1 that interacts with GILZ, thinking that overexpressing a small SGK1-related peptide that binds to GILZ might interfere with their interaction and block GILZ-mediated protection of SGK1 (FIG. 11A). Surprisingly, the interaction domain is not at the N-terminus of SGK1, but mapped to amino acids 122-157 of SGK1. An AdV was made encoding FLAG-SGK1(122-157), which we call SGK1-(Pep), to ectopically express it, finding that it could bind to GILZ in IP experiments, and that it blunted PE-mediated ANP expression consistent with a blockade of myocyte growth (FIG. 11C). Thus, it is anticipated that AAV9-FLAG-SGK1-(Pep) can increase SGK1 degradation and, thus, blunt pressure overload-induced pathological cardiac hypertrophy and subsequent heart failure in mice, in vivo.

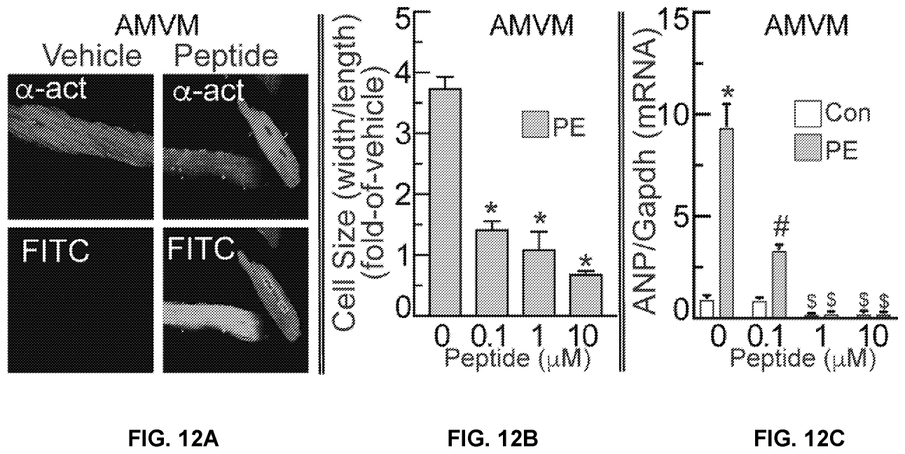

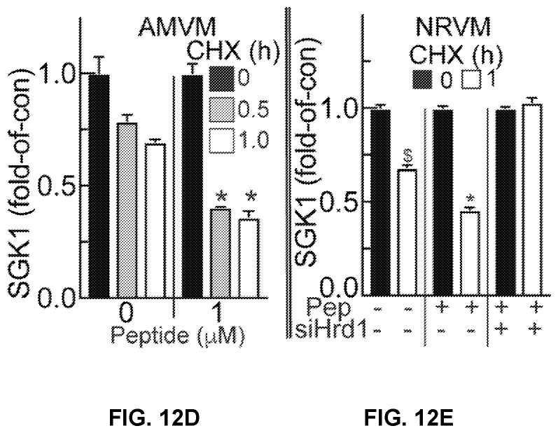

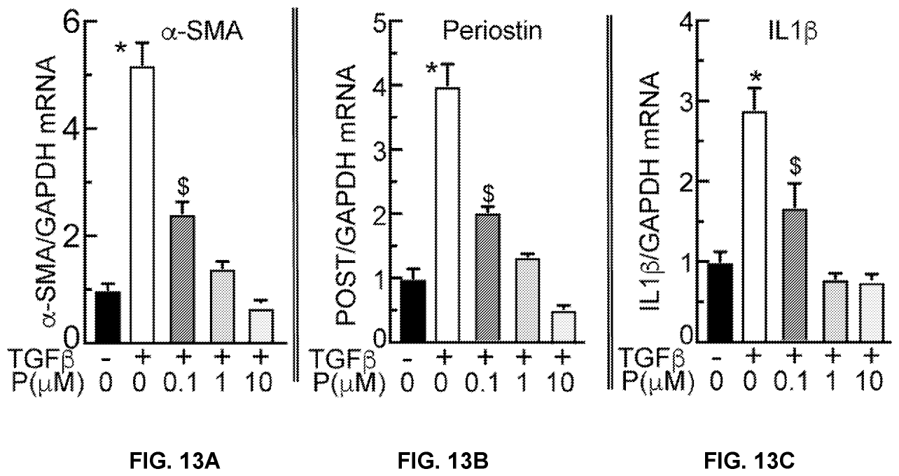

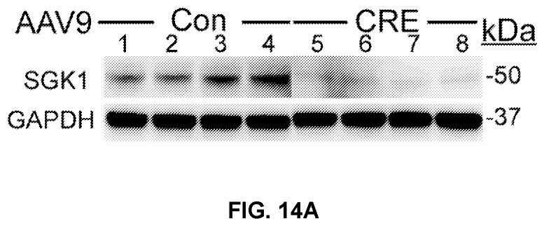

TAT-SGK1-(Pep) inhibits myocyte growth and fibroblast activation: Next, a SGK1-(Pep) peptide was designed and synthesized with an N-terminal TAT sequence to facilitate its direct delivery to cultured cells, and potentially to mice. To test its effectiveness, isolated adult mouse ventricular myocytes (AMVMs) were treated with either vehicle or a FITC-labeled form of the peptide, then imaged to show uptake (FIG. 12A). When AMVMs were treated±PE for 24 h, there was a TAT-SGK1-(Pep) dose-dependent decrease in cardiac myocyte remodeling and ANP mRNA (FIG. 12B, 12C). Thus, the TAT-SGK1-(Pep) affects AMVM growth as anticipated. TAT-SGK1-(Pep) was also found to accelerate endogenous SGK1 degradation in AMVM and NRVM (FIG. 12D, 12E). Importantly, in NRVM this degradation was dependent on Hrd1 (FIG. 12E), demonstrating that SGK1 is degraded by non-canonical ERAD in adult mouse cardiac myocytes. Finally, since TGFβ-stimulated SGK1 has been implicated in fibrosis (Artunc F and Lang F. Nephron Physiol. 2014 128:35-9), a major maladaptive aspect of pressure overload cardiac pathology, the effects of the TAT-SGK1-(Pep) on TGFβ-mediated induction of two markers of fibrosis, α-smooth muscle actin (α-SMA) and periostin, was examined in adult mouse ventricular fibroblasts (AMVF). The peptide effectively inhibited induction of both α-SMA and periostin over the same concentration range that it inhibited cardiac myocyte growth (FIG. 13A, 13B). Moreover, since TGFβ also induces an inflammatory response in cardiac fibroblasts (Gan W, et al. Biochim Biophys Acta Mol Basis Dis. 2018 1864:1-10), mRNA for IL1β, a marker of inflammasome activation, was measured showing that it was also inhibited by the peptide (FIG. 13C). These findings underscore the SGK1-specificity of the peptide, as well as the possibility that some of the adaptive effects of the peptide could include dampening inflammasome activation and decreasing fibrosis.

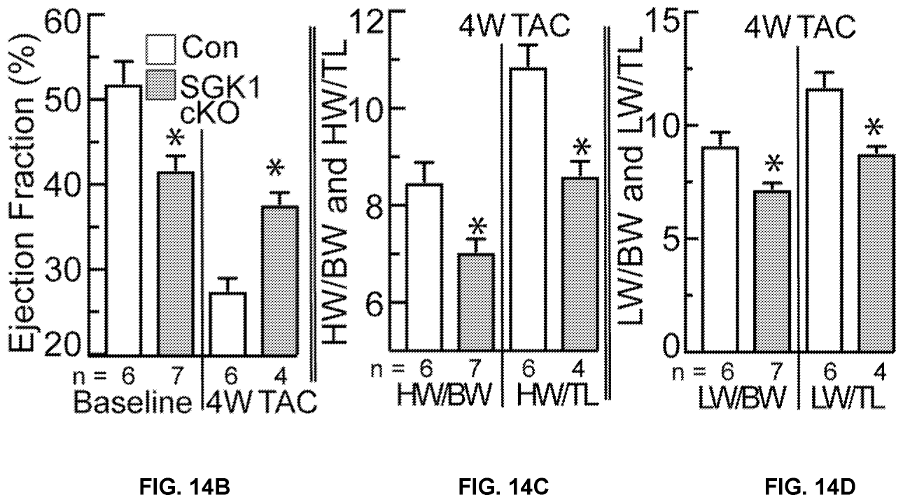

Hypertrophy and functional decline are blunted in SGK1 cKO mouse hearts: There have been no reports of studies using mice with cardiac-specific deletion of SGK1. Accordingly, SGK1^{fl/fl} mice were obtained and successful breeding set up (Fejes-Toth G, et al. Am J Physiol Renal Physiol. 2008 294:F1298-305). AAV9-Con or AAV9-MLC2v-CRE to SGK1^{fl/fl} were administered to mice, and after 14 d there was a clear decrease in endogenous SGK1 in CRE-treated mice (FIG. 14A). In a second group of mice, baseline echocardiography was performed, finding that the SGK1 cKO mice exhibited a slightly lower ejection fraction (EF) (FIG. 14B, Baseline), although the echo-based LV mass estimate for Con and SGK1 cKO mice was 87±7 mg and 80±5 mg, respectively, which were not significantly different, indicating similar starting heart weights. A study was performed on a small cohort of mice subjected to TAC. Four weeks after TAC, the EF in Con mice decreased from 52 to 27%, while the EF in SGK1 cKO mice started out lower, at 41%, but did not decrease significantly, ending at 38% (FIG. 14B, 4W TAC). After TAC, heart and lung weights (HW and LW) were lower in SGK1cKO mice (FIG. 14C, 14D). Thus, cardiac-specific deletion of SGK1, while not significantly affecting estimated heart weight slightly reduced baseline cardiac function and attenuated the decline in EF after 4W of TAC. Thus, SGK1 deletion decreased pressure overload-induced heart growth, as well as moderating the increase in lung weights, an indicator of decreased progression toward heart failure upon SGK1 deletion.

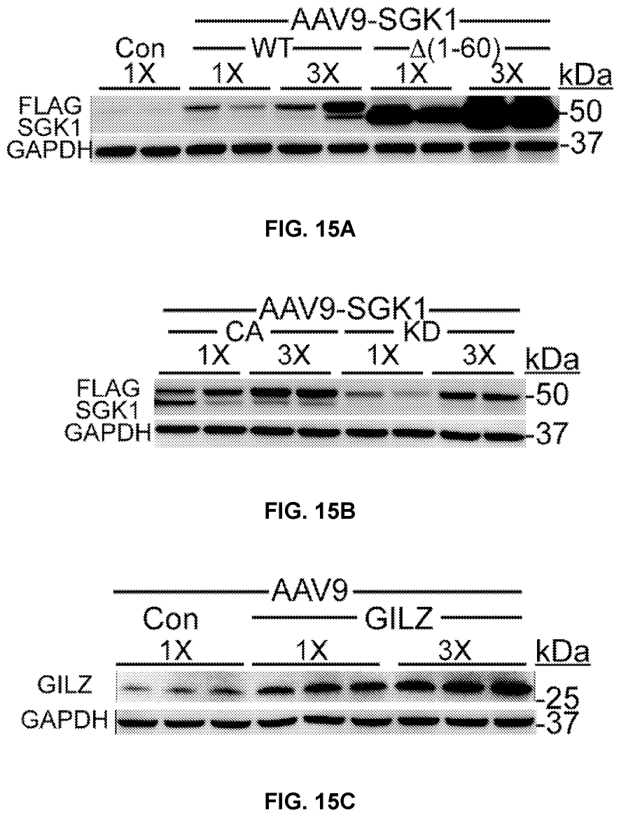

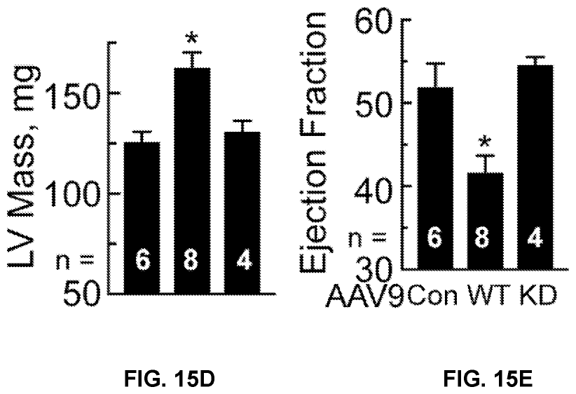

Generation of AAV9 for ectopic expression of SGK1 in mouse hearts: The effects of ectopic expression of various forms of SGK1 and wild type GILZ are examined in mouse cardiac myocytes, in vivo. For this purpose, AAV9 is used for efficient gene transfer to the heart as done previously to examine other genes in the heart, in vivo (Doroudgar S, et al. Circulation Research. 2015 117:536-546; Volkers M, et al. Proc Natl Acad Sci USA. 2013 110:12661-12666; Jin J K, et al. Circ Res. 2017 120:862-875; Blackwood E A, et al. Circ Res. 2019 124:79-93). a ventricular cardiac myocyte-specific promoter, MLC2v is used. AAV9-MLC2v FLAG-SGK1-WT, Δ(60), CA and KD, as well as GILZ (not FLAG tagged) have been generated. IB showed that each new virus supports expression of the intended proteins (FIG. 15A-15C). Two typical doses for each new virus was tested. Compared to the other SGK1 forms, FLAG-SGK1-Δ(60) is expressed at higher levels for a given dose of virus, most likely because it is not targeted to the ER and, therefore, as showed in NRVM, it is not degraded as rapidly as the other forms. A study was performed on a small cohort of mice treated with AAV9-Con, WT and KD, then 3 weeks later, subjected to TAC. Before TAC, echo revealed no significant cardiac functional differences between the virus treatments. However, two weeks after TAC, echo showed that estimated LV mass was increased significantly, while EF was decreased, only in the AAV9-SGK1-WT-treated mice (FIG. 15D, 15E), demonstrating that SGK1 exacerbated the effects of TAC and its kinase activity is required for this effect.

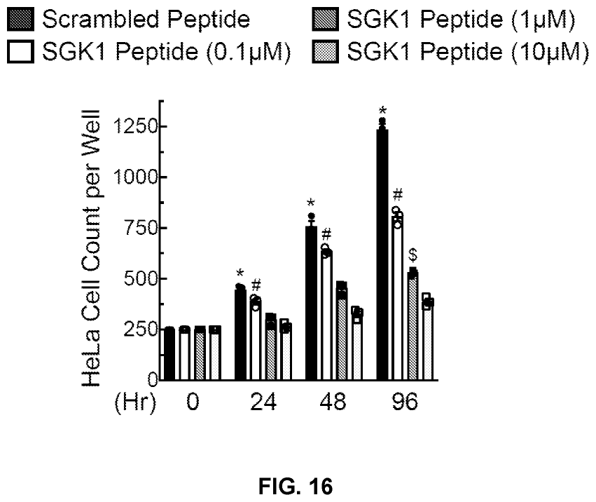

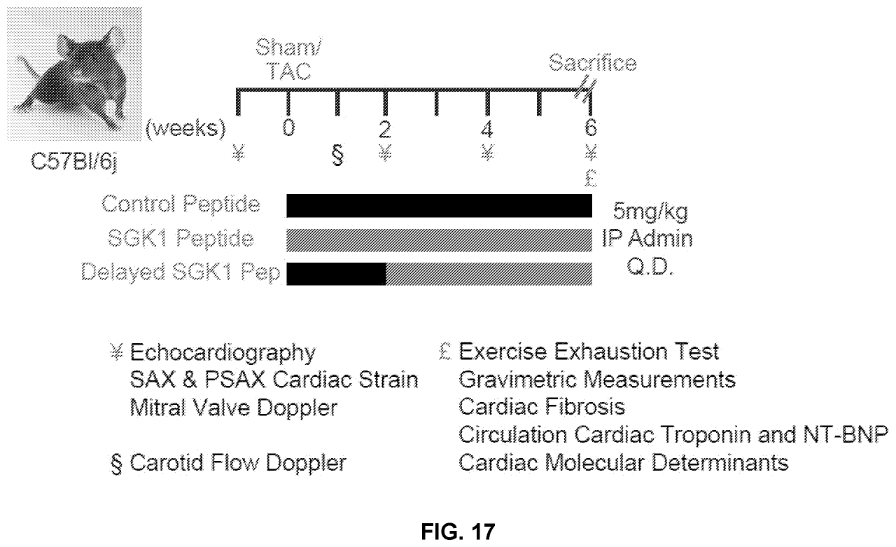

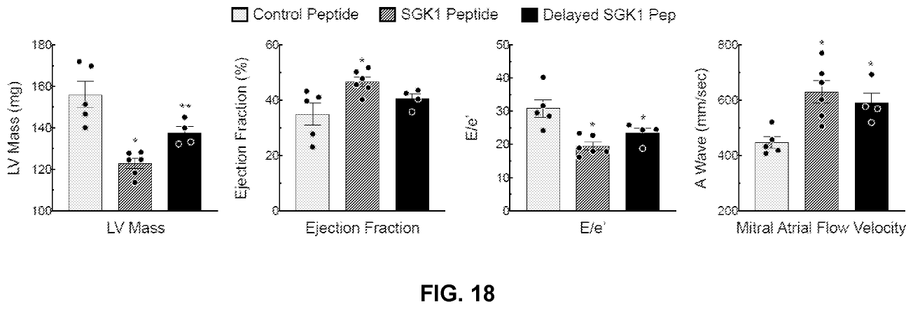

FIG. 16 shows immortalized cancer cells (HeLa) seeded at 250 cells/well on a 6-well plastic culture dish and treated with a scrambled peptide or the SGK1 peptide at concentrations of 0.1 μM, 1 μM, or 10 μM at timepoint 0 Hr. Cells were lifted off the dish and counted via hemocytomer after 24 Hr, 48 Hr, or 96 Hr in culture without refeeding during the course of the experiment.

### Example 2

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A fusion peptide comprising a peptide fragment of Serum/Glucocorticoid Regulated Kinase 1 (SGK1) and an internalization sequence,
wherein the peptide fragment comprises at least 10 consecutive amino acids of SEQ ID NO:1, or a variant thereof having at least 85% sequence identity to SEQ ID NO:1.

2. The fusion peptide of claim 1, wherein the peptide fragment of SGK1 comprises the amino acid sequence SEQ ID NO:1.

3. The fusion peptide of claim 1, wherein fusion peptide consists essentially of the amino acid sequence SEQ ID NO:1 and an internalization sequence.

4. The fusion peptide of claim 1, wherein peptide fragment of SGK1 comprises less than 200 consecutive amino acids from SEQ ID NO:2.

5. The fusion peptide of claim 1, wherein the internalization sequence comprises a HIV-TAT internalization domain.

6. The fusion peptide of claim 1, wherein the fusion peptide comprises the amino acid sequence SEQ ID NO:20.

7. The fusion peptide of claim 1, wherein the fusion peptide consists essentially of the amino acid sequence SEQ ID NO:20.

8. The fusion peptide of claim 1, further comprising a linker between the peptide fragment of SGK1 and the internalization sequence.

9. A method for treating a disease associated with aberrant Serum/Glucocorticoid Regulated Kinase 1 (SGK1) activity in a subject, comprising administering to the subject an agent that blocks the binding of endogenous SGK1 to endogenous glucocorticoid-induced leucine zipper (GILZ).

10. The method of claim 9, wherein the agent comprises a fusion peptide comprising a peptide fragment of Serum/Glucocorticoid Regulated Kinase 1 (SGK1) and an internalization sequence, wherein the peptide fragment comprises at least 10 consecutive amino acids of SEQ ID NO:1, or a variant thereof having at least 85% sequence identity to SEQ ID NO:1.

11. The method of claim 9, wherein the agent comprises an antibody or aptamer that specifically binds SEQ ID NO:1.

12. The method of claim 9, wherein the disease is a cancer.

13. The method of claim 12, wherein the cancer is a prostate cancer, colorectal carcinoma, glioblastoma, breast cancer, or endometrial cancer.

14. The method of claim 9, wherein the disease is a heart failure.


SEQUENCE LISTING (84 sequences)

SEQ ID NO: 1 - protein, 36 aa, Homo sapiens
VFYAVKVLQKKAILKKKEEKHIMSERNVLLKNVKHP

SEQ ID NO: 2 - protein, 431 aa, Homo sapiens
MTVKTEAAKGTLTYSRMRGMVAILIAFMKQRRMGLNDFIQKIANNSYACKHPEVQSILKISQPQEPELMNANPSPPPSPSQQINLGPSSNPHAKPSDFHFLKVIGKGSFGKVLLARHKAEEVFYAVKVLQKKAILKKKEEKHIMSERNVLLKNVKHPFLVGLHFSFQTADKLYFVLDYINGGELFYHLQRERCFLEPRARFYAAEIASALGYLHSLNIVYRDLKPENILLDSQGHIVLTDFGLCKENIEHNSTTSTFCGTPEYLAPEVLHKQPYDRTVDWWCLGAVLYEMLYGLPPFYSRNTAEMYDNILNKPLQLKPNITNSARHLLEGLLQKDRTKRLGAKDDFMEIKSHVFFSLINWDDLINKKITPPFNPNVSGPNDLRHFDPEFTEEPVPNSIGKSPDSVLVTASVKEAAEAFLGFSYAPPTDSFL

SEQ ID NO: 3 - protein, 9 aa, unidentified
RRRRRRRRR

SEQ ID NO: 4 - protein, 16 aa, unidentified
RQPKIWFPNRRKPWKK

SEQ ID NO: 5 - protein, 11 aa, unidentified
GRKKRRQRPPQ

SEQ ID NO: 6 - protein, 16 aa, unidentified
RQIKIWFQNRRMKWKK

SEQ ID NO: 7 - protein, 16 aa, unidentified
RQIAIWFQNRRMKWAA

SEQ ID NO: 8 - protein, 9 aa, unidentified
RKKRRQRRR

SEQ ID NO: 9 - protein, 21 aa, unidentified
TRSSRAGLQFPVGRVHRLLRK

SEQ ID NO: 10 - protein, 26 aa, unidentified
GWTLNSAGYLLGKINKALAALAKKIL

SEQ ID NO: 11 - protein, 18 aa, unidentified
KLALKLALKALKAALKLA

SEQ ID NO: 12 - protein, 16 aa, unidentified
AAVALLPAVLLALLAP

SEQ ID NO: 13 - protein, 10 aa, unidentified
VPMLKPMLKE

SEQ ID NO: 14 - protein, 28 aa, unidentified
MANLGYWLLALFVTMWTDVGLCKKRPKP

SEQ ID NO: 15 - protein, 18 aa, unidentified
LLIILRRRIRKQAHAHSK

SEQ ID NO: 16 - protein, 21 aa, unidentified
KETWWETWWTEWSQPKKKRKV

SEQ ID NO: 17 - protein, 18 aa, unidentified
RGGRLSYSRRRFSTSTGR

SEQ ID NO: 18 - protein, 15 aa, unidentified
SDLWEMMMVSLACQY

SEQ ID NO: 19 - protein, 12 aa, unidentified
TSPLNIHNGQKL

SEQ ID NO: 20 - protein, 48 aa, synthetic construct
GRKKRRQRRRPQVFYAVKVLQKKAILKKKEEKHIMSERNVLLKNVKHP

SEQ ID NO: 21 - protein, 35 aa, Homo sapiens
FYAVKVLQKKAILKKKEEKHIMSERNVLLKNVKHP

SEQ ID NO: 22 - protein, 34 aa, synthetic construct
YAVKVLQKKAILKKKEEKHIMSERNVLLKNVKHP

SEQ ID NO: 23 - protein, 33 aa, synthetic construct
AVKVLQKKAILKKKEEKHIMSERNVLLKNVKHP

SEQ ID NO: 24 - protein, 32 aa, Homo sapiens
VKVLQKKAILKKKEEKHIMSERNVLLKNVKHP

SEQ ID NO: 25 - protein, 31 aa, Homo sapiens
KVLQKKAILKKKEEKHIMSERNVLLKNVKHP

SEQ ID NO: 26 - protein, 30 aa, Homo sapiens
VLQKKAILKKKEEKHIMSERNVLLKNVKHP

SEQ ID NO: 27 - protein, 29 aa, Homo sapiens
LQKKAILKKKEEKHIMSERNVLLKNVKHP

SEQ ID NO: 28 - protein, 28 aa, Homo sapiens
QKKAILKKKEEKHIMSERNVLLKNVKHP

SEQ ID NO: 29 - protein, 27 aa, Homo sapiens
KKAILKKKEEKHIMSERNVLLKNVKHP

SEQ ID NO: 30 - protein, 34 aa, Homo sapiens
FYAVKVLQKKAILKKKEEKHIMSERNVLLKNVKH

SEQ ID NO: 31 - protein, 33 aa, Homo sapiens
FYAVKVLQKKAILKKKEEKHIMSERNVLLKNVK

SEQ ID NO: 32 - protein, 32 aa, Homo sapiens
FYAVKVLQKKAILKKKEEKHIMSERNVLLKNV

SEQ ID NO: 33 - protein, 31 aa, Homo sapiens
FYAVKVLQKKAILKKKEEKHIMSERNVLLKN

SEQ ID NO: 34 - protein, 30 aa, Homo sapiens
FYAVKVLQKKAILKKKEEKHIMSERNVLLK

SEQ ID NO: 35 - protein, 29 aa, Homo sapiens
FYAVKVLQKKAILKKKEEKHIMSERNVLL

SEQ ID NO: 36 - protein, 28 aa, Homo sapiens
FYAVKVLQKKAILKKKEEKHIMSERNVL

SEQ ID NO: 37 - protein, 27 aa, Homo sapiens
FYAVKVLQKKAILKKKEEKHIMSERNV

SEQ ID NO: 38 - protein, 26 aa, Homo sapiens
FYAVKVLQKKAILKKKEEKHIMSERN

SEQ ID NO: 39 - protein, 33 aa, Homo sapiens
YAVKVLQKKAILKKKEEKHIMSERNVLLKNVKH

SEQ ID NO: 40 - protein, 32 aa, Homo sapiens
YAVKVLQKKAILKKKEEKHIMSERNVLLKNVK

SEQ ID NO: 41 - protein, 31 aa, Homo sapiens
YAVKVLQKKAILKKKEEKHIMSERNVLLKNV

SEQ ID NO: 42 - protein, 30 aa, Homo sapiens
YAVKVLQKKAILKKKEEKHIMSERNVLLKN

SEQ ID NO: 43 - protein, 29 aa, Homo sapiens
YAVKVLQKKAILKKKEEKHIMSERNVLLK

SEQ ID NO: 44 - protein, 28 aa, Homo sapiens
YAVKVLQKKAILKKKEEKHIMSERNVLL

SEQ ID NO: 45 - protein, 27 aa, Homo sapiens
YAVKVLQKKAILKKKEEKHIMSERNVL

SEQ ID NO: 46 - protein, 26 aa, Homo sapiens
YAVKVLQKKAILKKKEEKHIMSERNV

SEQ ID NO: 47 - protein, 32 aa, Homo sapiens
AVKVLQKKAILKKKEEKHIMSERNVLLKNVKH

SEQ ID NO: 48 - protein, 31 aa, Homo sapiens
AVKVLQKKAILKKKEEKHIMSERNVLLKNVK

SEQ ID NO: 49 - protein, 30 aa, Homo sapiens
AVKVLQKKAILKKKEEKHIMSERNVLLKNV

SEQ ID NO: 50 - protein, 29 aa, Homo sapiens
AVKVLQKKAILKKKEEKHIMSERNVLLKN

SEQ ID NO: 51 - protein, 28 aa, Homo sapiens
AVKVLQKKAILKKKEEKHIMSERNVLLK

SEQ ID NO: 52 - protein, 27 aa, Homo sapiens
AVKVLQKKAILKKKEEKHIMSERNVLL

SEQ ID NO: 53 - protein, 26 aa, Homo sapiens
AVKVLQKKAILKKKEEKHIMSERNVL

SEQ ID NO: 54 - protein, 31 aa, Homo sapiens
VKVLQKKAILKKKEEKHIMSERNVLLKNVKH

SEQ ID NO: 55 - protein, 30 aa, Homo sapiens
VKVLQKKAILKKKEEKHIMSERNVLLKNVK

SEQ ID NO: 56 - protein, 29 aa, Homo sapiens
VKVLQKKAILKKKEEKHIMSERNVLLKNV

SEQ ID NO: 57 - protein, 28 aa, Homo sapiens
VKVLQKKAILKKKEEKHIMSERNVLLKN

SEQ ID NO: 58 - protein, 27 aa, Homo sapiens
VKVLQKKAILKKKEEKHIMSERNVLLK

SEQ ID NO: 59 - protein, 26 aa, Homo sapiens
VKVLQKKAILKKKEEKHIMSERNVLL

SEQ ID NO: 60 - protein, 30 aa, Homo sapiens
KVLQKKAILKKKEEKHIMSERNVLLKNVKH

SEQ ID NO: 61 - protein, 29 aa, Homo sapiens
KVLQKKAILKKKEEKHIMSERNVLLKNVK

SEQ ID NO: 62 - protein, 28 aa, Homo sapiens
KVLQKKAILKKKEEKHIMSERNVLLKNV

SEQ ID NO: 63 - protein, 27 aa, Homo sapiens
KVLQKKAILKKKEEKHIMSERNVLLKN

SEQ ID NO: 64 - protein, 26 aa, Homo sapiens
KVLQKKAILKKKEEKHIMSERNVLLK

SEQ ID NO: 65 - protein, 29 aa, Homo sapiens
VLQKKAILKKKEEKHIMSERNVLLKNVKH

SEQ ID NO: 66 - protein, 28 aa, Homo sapiens
VLQKKAILKKKEEKHIMSERNVLLKNVK

SEQ ID NO: 67 - protein, 27 aa, Homo sapiens
VLQKKAILKKKEEKHIMSERNVLLKNV

SEQ ID NO: 68 - protein, 26 aa, Homo sapiens
VLQKKAILKKKEEKHIMSERNVLLKN

SEQ ID NO: 69 - protein, 28 aa, Homo sapiens
LQKKAILKKKEEKHIMSERNVLLKNVKH

SEQ ID NO: 70 - protein, 27 aa, Homo sapiens
LQKKAILKKKEEKHIMSERNVLLKNVK

SEQ ID NO: 71 - protein, 26 aa, Homo sapiens
LQKKAILKKKEEKHIMSERNVLLKNV

SEQ ID NO: 72 - protein, 27 aa, Homo sapiens
QKKAILKKKEEKHIMSERNVLLKNVKH

SEQ ID NO: 73 - protein, 26 aa, Homo sapiens
QKKAILKKKEEKHIMSERNVLLKNVK

SEQ ID NO: 74 - protein, 26 aa, Homo sapiens
KKAILKKKEEKHIMSERNVLLKNVKH

SEQ ID NO: 75 - protein, 5 aa, unidentified
GGGGS

SEQ ID NO: 76 - protein, 9 aa, unidentified
GGGGGGGGG

SEQ ID NO: 77 - protein, 9 aa, unidentified
QQQQQQQQQ

SEQ ID NO: 78 - protein, 9 aa, unidentified
SSSSSSSSS

SEQ ID NO: 79 - protein, 9 aa, unidentified
GGGGGCPPC

SEQ ID NO: 80 - protein, 21 aa, unidentified
GGGGSSSGGGGSSSGGGGSSS

SEQ ID NO: 81 - protein, 13 aa, unidentified
SCVPLMRCGGCCN

SEQ ID NO: 82 - protein, 14 aa, unidentified
PSCVPLMRCGGCCN

SEQ ID NO: 83 - protein, 9 aa, unidentified
GDLIYRNQK

SEQ ID NO: 84 - protein, 23 aa, unidentified
GGGGGGGGGPSCVPLMRCGGCCN